(19) United States
(12) Patent Application Publication
(10) Pub. No.: US 2014/0051918 A1
(43) Pub. Date: Feb. 20, 2014

(54) LAPAROSCOPIC GASTRIC BAND WITH ACTIVE AGENTS

(71) Applicant: Allergan, Inc., Irvine, CA (US)

(72) Inventors: Joseph S. Raven, Tigard, OR (US); Janel A. Birk, Oxnard, CA (US)

(73) Assignee: Allergan, Inc., Irvine, CA (US)

(21) Appl. No.: 14/063,781

(22) Filed: Oct. 25, 2013

Related U.S. Application Data
(63) Continuation of application No. 13/024,780, filed on Feb. 10, 2011, now abandoned.
Application No. 13/024,780 is a continuation-in-part of application No. 12/771,671, filed on Apr. 30, 2010.
(60) Provisional application No. 61/174,874, filed on May 1, 2009.

(51) Int. Cl. A61F 5/00

(52) U.S. Cl. CPC A61F 5/0063; A61F 5/0003; A61F 5/0026
USPC 600/37; 604/500

(57) ABSTRACT

A gastric banding system is provided which generally includes a gastric band and an active agent, for example, a metabolic agent or satiety inducing agent. The band may be structured to contain the agent and permit controlled release of the agent to the patient while the band is positioned around the stomach. Methods for treating obesity are also provided which include positioning a gastric band on the stomach of a patient and administering a satiety inducing agent to the patient while the gastric band is positioned on the stomach. In one embodiment, the active agent may be contained in a reservoir and dispensed to a portion of the patient's body.

### RELATED APPLICATIONS

This application is a continuation of U.S. patent application Ser. No. 13/024,780, filed Feb. 10, 2011, which is a continuation-in-part of U.S. patent application Ser. No. 12/771,671, filed on Apr. 30, 2010, which claims the benefit of and priority to U.S. Provisional Patent Application No. 61/174,874, filed on May 1, 2009. The entire contents of each of these applications are hereby incorporated by reference herein.

### BACKGROUND

The present invention relates to laparoscopic gastric banding for treatment of obesity and obesity related disorders and more specifically relates to a laparoscopic gastric band system including active agents.

Laparoscopic adjustable gastric bands have a successful history of inducing weight loss in obese patients. The band is secured around the stomach just below the gastroesophageal junction. This creates a small pouch above the band which can only accept a small volume of food. Generally, this allows the patient to ingest only a small amount of food before the patient begins to feel satiated and full, and consequently, the patient is less likely to eat to excess. With reduced caloric intake, the patient loses weight. It is known that some patients, however, reach a “plateau” in their rate of weight loss over time, even with the gastric band in place.

Despite the relative safety and success of gastric banding in treating obesity and obesity related conditions, there remains a need for improved systems and methods for treating obesity and obesity related conditions in some patients.

### SUMMARY

The present invention provides a gastric banding system generally comprising a gastric band structured to be placed around the stomach of a patient. Further, the band is capable of dispensing an active agent, such as but not limited to, a metabolic agent, for example, a satiety inducing agent, to the patient while the band is positioned around the stomach. The system may provide more effective obesity treatment relative to obesity treatment using a gastric band alone.

For example, the system may further comprise a metabolic agent, or a satiety inducing agent, for being dispensed to the patient while the band is positioned around the stomach. The satiety inducing agent may be incorporated into the gastric band.

In one embodiment, an ancillary device is incorporated into the gastric band and the ancillary device includes, or is capable of dispensing to the patient, a satiety inducing agent. The ancillary device may be structured to provide controlled release of the satiety inducing agent to the patient.

For example, the ancillary device comprises a membrane or film permeable to a satiety inducing agent. The agent may be covered or enclosed by the membrane and is released into the body by diffusion through the membrane.

In other embodiments, the ancillary device may comprise a composition including a matrix material and a satiety inducing agent combined with the matrix material. The matrix material may be a biodegradable or bioerodible material, for example a bioerodible polymer which, during erosion thereof in the body, releases the agent from the composition in a controlled manner.

Alternatively, the ancillary device may be a non-bioerodible material. The device may include structures for containing and releasing the satiety inducing agents, for example in a controlled manner. In one embodiment, the ancillary device includes recessions, pores or grooves capable of containing a satiety inducing agent.

In some embodiments of the invention, the satiety inducing agent is a hormone, for example a peptide hormone. The peptide hormone may be at least one agent selected from the group consisting of Glucagon-like peptide (GLP-1), Oxyntomodulin (OXM), Peptide YY (PYY), Pancreatic Polypeptide (PP), Insulin, Leptin, Gastrin, Ghrelin blocker, inhibitors of DPP-IV, and Amylin. The satiety inducing agent may be Cholecystokinin (CCK), which may suppress appetite when administered with or without gastric distension.

In other embodiments the ancillary device further includes a film or membrane in contact with the agent and capable of releasing the agent from the ancillary device and into the patient, for example, at a controlled rate.

In some embodiments, the gastric band itself is structured to be capable of releasing a satiety inducing agent into the patient at a controlled rate.

The present invention further provides a method of treating obesity or an obesity related condition in a patient. In one embodiment, the method comprises implanting a gastric band in a patient and providing a composition effective to induce satiety in the patient wherein the composition is positioned between the gastric band and the stomach of the patient when the gastric band is so positioned around the stomach of the patient.

For example, the composition may comprise compositions as described elsewhere herein. For example, the composition may include a satiety inducing agent and a bioerodible material combined with the agent wherein the agent is distributed in the bioerodible material and is effective, when released into the patient, to at least assist in inducing satiety in the patient.

In another aspect of the invention, a method for treating obesity or an obesity related condition is provided wherein the method comprises positioning a gastric band on the stomach of a patient and administering a satiety inducing agent to the patient while the gastric band is positioned on the stomach.

The step of administering may comprise dispensing the agent to one of the stomach, intestine, peritoneum, intra-peritoneal cavity, and abdomen of the patient. In other embodiments, the agent is administered subcutaneously to the patient. In yet other embodiments, the step of administering comprises administering the agent directly to the central nervous system. In yet other embodiments, the agent is administered as an inhalant.

The step of administering may further comprise controlling a rate of release of the agent into the patient.

The agent may be administered at a controlled rate over a period of at least about six months, or at least about one year or at least about three years. In some embodiments, the controlled rate includes a period of dosage tapering, or a period of dosage increasing.

It is to be appreciated that the active agents useful in the present invention are not limited to satiety inducing agents but may also include any active agents, for example, other metabolic agents, that may provide some benefit to a patient suffering from obesity and/or obesity related conditions.

In one embodiment, the present invention comprises an implantable system for the treatment of obesity, including a reservoir configured to be laparoscopically implanted into a patient's body and contain an active agent being effective, when released into the patient, to at least assist in effecting weight loss in the patient. The reservoir has an outlet configured to allow the active agent to exit the reservoir and contact a portion of the patient's body. A tube may be coupled to the reservoir to allow the active agent to be distributed to a desired portion of the patient's body. The reservoir may be positioned laparoscopically around the patient's stomach, or elsewhere as desired in the patient's body.

In one embodiment, the present invention comprises a method for the treatment of obesity, comprising the step of implanting a reservoir into a patient's body laparoscopically. The reservoir is configured to contain an active agent being effective, when released into the patient, to at least assist in effecting weight loss in the patient. The reservoir has an outlet configured to allow to active agent to exit the reservoir and contact a portion of the patient's body. The reservoir may be coupled to a gastric band, which is implanted into the patient's body along with the reservoir.

In one embodiment, the present invention comprises a method for the treatment of obesity comprising the steps of inserting an electrode into a patient's body laparoscopically and coupling the electrode to the lower third of the patient's esophagus. The electrode is configured to apply electric stimulation to the lower third of the patient's esophagus. The electrode is utilized in combination with a gastric band positioned around a portion of the patient's stomach to form a stoma. The electrode may be coupled to the gastric band.

In one embodiment, the present invention comprises a gastric banding system for the treatment of obesity comprising a gastric band configured to encircle a portion of a patient's stomach to form a stoma, an implantable sensor configured to sense a biological characteristic of the patient, and an external control device configured to receive a signal sent in response to a biological characteristic sensed by the sensor, and to produce a notification in response to the signal for a user to perform an action effective to vary the biological characteristic sensed by the sensor. The biological characteristic may comprise a hormone level of the patient. The action may comprise injection of an active agent into the patient's body, inhaling of an active agent by the patient, drinking of an active agent by the patient, application of a patch to the patient's body being capable of distributing an active agent to the patient, spraying of an active agent into the patient's mouth, swallowing of a pill by the patient containing an active agent, insertion of a gum or film containing an active agent into the patient's mouth. A combination of actions may be taken, in response to the biological characteristic sensed by the sensor.

Each and every feature described herein, and each and every combination of two or more of such features, is included within the scope of the present invention provided that the features included in such a combination are not mutually inconsistent.

### BRIEF DESCRIPTION OF THE DRAWINGS

FIG. 1 is a perspective view of a system for treating obesity and obesity related conditions, in accordance with the present invention.

FIGS. 2A and 2B are perspective views of surface structures useful for containing active agents in conjunction with a gastric band, in accordance with systems of the present invention.

FIG. 3 is a simplified representation of a diffusion material useful for controlling release of active agents in conjunction with a gastric band, in accordance with systems of the present invention.

FIG. 4 is a perspective view of a system for the treatment of obesity, according to an embodiment of the present invention.

FIG. 5 is a perspective view of a reservoir and gastric band, according to an embodiment of the present invention.

FIG. 6 is a perspective view of a reservoir and gastric band, according to an embodiment of the present invention.

FIG. 7 is a perspective view of a system for the treatment of obesity, according to an embodiment of the present invention.

FIG. 8 is a perspective view of a reservoir, according to an embodiment of the present invention.

FIG. 9 is a perspective view of a reservoir and gastric band, according to an embodiment of the present invention.

FIG. 10 is a perspective view of a reservoir and gastric band, according to an embodiment of the present invention.

FIG. 11 is a perspective view of a reservoir, according to an embodiment of the present invention.

FIG. 12 is a schematic view of a reservoir, according to an embodiment of the present invention.

FIG. 13 is a schematic view of a reservoir, according to an embodiment of the present invention.

FIG. 14 is a flowchart representing an exemplary method for the treatment of obesity, according to an embodiment of the present invention.

FIG. 15 is a perspective view of a system for the treatment of obesity, according to an embodiment of the present invention.

FIG. 16 is a perspective view of a gastric band, according to an embodiment of the present invention.

FIG. 17 is a perspective view of a system for the treatment of obesity, according to an embodiment of the present invention.

FIG. 18 is a flowchart representing an exemplary method for the treatment of obesity, according to an embodiment of the present invention.

FIG. 19 is a perspective view of a system for the treatment of obesity, according to an embodiment of the present invention.

### DETAILED DESCRIPTION

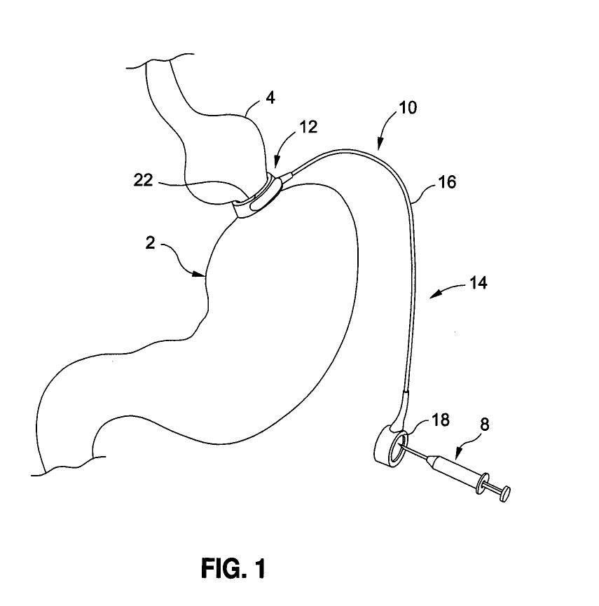

Turning now to FIG. 1, the present invention provides a gastric band system 10 which is structured to dispense an active agent, for example, a metabolic agent, for example a satiety inducing agent, for example, a satiety gut hormone or bioactive molecule, into the body. Although the present disclosure will typically be discussing, specifically, satiety inducing agents, it is to be appreciated that the present invention, in all embodiments, is not limited to active agents that are, specifically, satiety inducing agents. Active agents useful with the present invention are intended to include other compositions, drugs or other agents, for example, agents that affect body metabolism without necessarily affecting satiety, that are believed to be effective, at least to some degree, in facilitating weight loss in a human being. In all embodiments discussed throughout this application, the active agent is selected to at least assist in effecting weight loss in a patient.

In an exemplary embodiment, the system 10 generally comprises a gastric band 12 which is structured to be placed at the stomach 2 of a patient in such a manner so as to form a stoma 4, or pouch. The gastric band 12 may be an inflatable hydraulic gastric band (such as shown) or a mechanically adjustable gastric band, for example, a mechanically adjustable gastric band operated by a motor. The gastric band 12 may include a stoma adjustment mechanism 14, comprising, for example, a fill line 16 and an implantable access port 18. By injecting or withdrawing a filling fluid from access port 18, for example, through the use of a needle/syringe 8, a physician can adjust a level of restriction of the band 12.

Further, the system 10 is capable of dispensing an active agent, for example, but not limited to, a satiety inducing agent, to the patient while the band is positioned around the stomach 2. The system 10 may provide more effective obesity treatment relative to obesity treatment using a gastric band alone.

For example, the system 10 may further comprise an active agent for being dispensed to the patient while the gastric band 12 is positioned at or around the stomach 2. The active agent may be incorporated into the gastric band 12.

In some embodiments, the system 10 further comprises an ancillary device 22 capable of dispensing to the patient, an active agent, while the system 10 is implanted in the patient. The ancillary device 22 may be incorporated into the gastric band 12, for example, at a region of the band 12 in contact with the stomach 2.

In some embodiments, the ancillary device comprises a composition incorporated into the gastric band. The composition may comprise a matrix material and an active agent, such as a satiety inducing agent, combined with the matrix material. The matrix material may be a biodegradable material, referred to equivalently as a bioerodible material, for example, a bioerodible polymer which, during erosion thereof in the body, releases the agent from the composition, for example, in a controlled manner, for example in a time-release fashion.

Alternatively, the ancillary device may comprise a non-bioerodible material structured to facilitate release of an active agent into the body. In some embodiments, the device includes structures for containing and releasing active agents, for example, in a controlled manner. Combinations of bioerodible and non-bioerodible materials for containing and releasing active agents are also contemplated.

In one embodiment, the ancillary device includes recessions, pores or grooves capable of containing an agent.

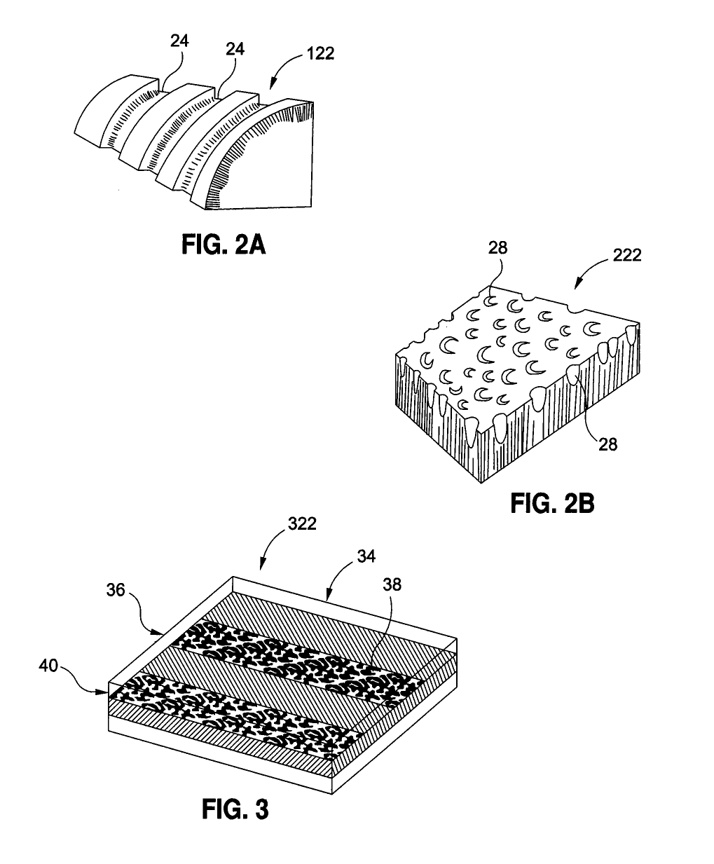

For example, an ancillary device 122, useful in the present systems, is shown FIG. 2A. The ancillary device 122 may include one or more of the features of the ancillary device 22 described elsewhere herein.

The ancillary device 122 comprises a polymer surface having one or more indentations or grooves 24 capable of containing or holding a satiety inducing agent, or a composition containing a satiety inducing agent, for example, in solid, gel, powder, paste or other form.

Turning now to FIG. 2B, alternatively or additionally, the ancillary device 222 comprises a polymer surface having a porous or other irregular structure, wherein the pores 28 are capable of containing or holding an agent, or a composition such as a matrix material containing an agent.

The ancillary devices 22, 122, 222 may be made of any suitable, biocompatible material, for example, any suitable material approved by the Food and Drug Administration (FDA) for use in humans, for example, as approved for long term administration of agents and long term placement in the body. In one embodiment, the material is ethylene vinyl acetate (EVA).

In some embodiments, the active agent is a satiety inducing agent, for example, a hormone, for example a peptide hormone. The peptide hormone may be at least one agent selected from a group consisting of Glucagon-like peptide (GLP-1), Oxyntomodulin (OXM), Peptide YY (PYY) and Peptide YY (3-36) (PYY (3-36)), Pancreatic Polypeptide (PP), Insulin, Leptin, Gastrin, Ghrelin blocker, inhibitors of DPP-IV, and Amylin. The satiety inducing agent may be Cholecystokinin (CCK) and Cholecystokinin 8 (CCK-8).

In some embodiments of the invention, the active agent is an agent selected from a list of agents consisting of Glial-Derived Neurotrophic Factor (GDNF); Serotonin; Dopamine and its Analogues such as: Ibogaine, Noribogaine, 18-MC, and Cabergoline; Ciliary-derived Neurotrophic Factor (CNTF); Cocaine-Amphetamine Regulated Transcript (CART); Serotonin and its Analogues; Gastric Inhibitory Peptide or Glucose-dependant Insulinotropic Peptide (GIP); Neuropeptide Y (NPY) receptor antagonists and iRNA/siRNA; Orexin A and B receptor antagonists and iRNA/siRNA; Agouti Related Peptide (AgRP) receptor antagonists and iRNA/siRNA; Cannabinoid receptor antagonists and iRNA/siRNA; the Melanocortins: Pro-Opiomelanocortin (POMC), Alpha and Beta Melanocyte Stimulating Hormone (α and β MSH); Melanin Concentrating Hormone (MCH) receptor antagonists and iRNA/siRNA; Adenosine Mono-Phosphate activated protein Kinase (AMPK); 5-aminoimidazole-4-carboxamide-1-β-D-ribofuranoside (AICAR); and Peroxisome Proliferator-Activated Receptor Delta Agonist (PPARδ-agonist).

Discussions of gastrointestinal hormones that control appetite can be found in Chaudhri. O. B., Wynne, K., and Bloom, S. R. 2008. Can gut hormones control appetite and prevent obesity?. Diabetes Care 31 (Suppl. 2): s284-s289 and Cummings, D. E. and Overduin, J. 2007. Gastrointestinal regulation of food intake. J. Clin. Invest. 117: 13-23, the entire disclosures of which are incorporated herein by reference.

In other embodiments of the invention, the active agent may be any suitable active agent that improves the weight-loss effect of the gastric band. For example, the active agent may be an agent that affects metabolism of a patient independently of the effect, if any, on satiety of the patient. Metabolic agents that are known or suspected to have a positive effect on weight loss are known to those of skill in the art.

Any of the active agents discussed throughout this application may be bioengineered to resist the breakdown of the active agent. For example, in an embodiment in which the active agent comprises a hormone, enzymes within a patient's body will begin a process of breaking down and rendering the hormone ineffective after the hormone is introduced into the patient's body. The enzymes target specific sites, particularly amino acids of the hormone, to cleave the hormone molecule, thus inactivating the hormone by changing its ability to bind to its receptor or exert its intended effect. To prevent this undesirable result, specific DNA capable of producing the hormone may be identified and modified to reduce the enzymatic degradation. For example, once a specific DNA sequence has been isolated and identified for the hormone of interest, small changes can be made to the DNA coding sequence. By altering the amino acid that is expressed following post-translational processing, insignificant changes can be made to the hormone molecule's stereo-structure while making the hormone relatively resistant to enzymatic degradation, thereby extending its half-life and efficacy. Methods of producing recombinant DNA are discussed in “AN INTRODUCTION TO GENETIC ANALYSIS” by Anthony Griffiths, Jeffery Miller, David Suzuki, Richard Lewontin, and William Gelbert, the entirety of which is incorporated by reference. Further information may also be found in “MOLECULAR CELL BIOLOGY” by Harvey Lodish, Arnold Berk, Paul Matsudaira, Chris Kaiser, Monty Krieger, Matthew Scott, S. Lawrence Zipursky, and James Darnell, the entirety of which is incorporated by reference.

In one embodiment, a Phenylethylene glycol (PEG) group may be added to any of the active agents discussed throughout this application, to enhance the effectiveness and longevity of the agent.

Referring now as well to FIG. 3, in some embodiments of the invention, the ancillary device 22 comprises a film or membrane 322 which makes up a surface of the gastric band 12, for example, a surface of the band which contacts the stomach when the band is appropriately positioned. In one embodiment, the film 322 forms at least a portion of an inner circumferential surface of the gastric band 12. The film is capable of releasing a satiety inducing agent from the band and into the patient, for example, at a controlled rate.

For example, the film 322 may comprise a first membrane layer 34 and a second membrane layer 36. The film 322 may further comprise a composition containing a satiety inducing agent, wherein the composition is located adjacent, for example, between the first and second membrane layers 34, 36. The first and second membrane layers 34, 36 may comprise EVA or other suitable polymer or copolymer.

In the shown embodiment, the film 322 further comprises first and second agent layers 38, 40 which are made up of a composition containing a satiety inducing agent. The first and second agent layers 38, 40 are disposed in an alternating fashion with respect to the first and second membrane layers 34, 36. The membrane layers 34, 36 may have a known diffusion rate relative to the selected satiety inducing agent.

The film 322 is effective to control dosage and delivery of the agents to the patient. The film 322 may therefore have a desired porosity and/or be made of a suitable material so as to provide a controlled release of the agent.

For example, each of the ancillary devices described herein, for example, devices 122, 222 and 322, may be structured to provide effective concentrations of the agent for about six months, or for about one year, about two years, or about three years or more. In some embodiments, the devices 122, 222, 322 are structured to provide a sustained release rate, for example, of three years followed by a gradually decreasing release rate over the next about two to about three years. The duration of the effective concentration of the agent, and the release rate, may be varied as desired. Numerous release protocols are contemplated by the inventors, and are understood to fall within the scope of the present invention.

The present invention further provides a method of treating obesity or an obesity related condition in a patient. In one embodiment, the method comprises implanting a gastric band in a patient and providing a composition effective to induce satiety in the patient wherein the composition is positioned between the gastric band and the stomach of the patient when the gastric band is so positioned around the stomach of the patient.

For example, the composition may comprise a composition as described elsewhere herein. For example, the composition may include a satiety inducing agent and a bioerodible material combined with the agent wherein the agent is distributed in the bioerodible material and is effective, when released into the patient, to at least assist in inducing satiety in the patient.

In another aspect of the invention, a method for treating obesity or an obesity related condition is provided wherein the method comprises positioning a gastric band on the stomach of a patient and administering a satiety inducing agent to the patient while the gastric band is positioned on the stomach.

The step of administering may comprise dispensing the agent to one of the stomach, intestine, peritoneum, intra-peritoneal cavity, and abdomen of the patient. In other embodiments, the agent is administered subcutaneously to the patient. In yet other embodiments, the step of administering comprises administering the agent directly to the central nervous system. In yet other embodiments, the agent is administered as an inhalant.

The step of administering may further comprise controlling a rate of release of the agent into the patient.

The agent may be administered at a controlled rate over a period of at least about six months, or at least about one year or at least about three years. In some embodiments, the controlled rate includes a period of dosage tapering, or a period of dosage increasing.

Exemplary peptide hormones which, alone or in combination, can be used in accordance with the invention include Glucagon-like peptide (GLP-1), Oxyntomodulin (OXM), Peptide YY (PYY), Pancreatic Polypeptide (PP), Amylin, Leptin, Gastrin or Ghrelin blocker. Another hormone that suppresses appetite when administered with or without gastric distension is Cholecystokinin (CCK), and other brain-gut satiety hormones such as Pro-opiomelanocortin (POMC), or others, or any combination of the above.

In the publication, “Can Gut Hormones Control Appetite and Prevent Obesity?” by Chaudhri, et al, research conducted on Ghrelin, GLP-1, Oxyntomodulin, Inhibitors of DPP-IV, Amylin, Peptide YY, and Pancreatic Polypeptide to control appetite, are described. These as well as other hormones may be useful in accordance with the present invention. Similarly, “Gastrointestinal Regulation of Food Intake” by David E. Cummings et al describes the efficacy of satiety hormones to boost weight loss.

The agent could also be applied to the band via a slow release drug eluting coating similar to coatings used on cardiovascular stents such as the Cordis Sirolimus Drug eluting stent or the contraceptive device Norplant. The coating could be applied directly to the band 12 for a slow release of the drug into the body.

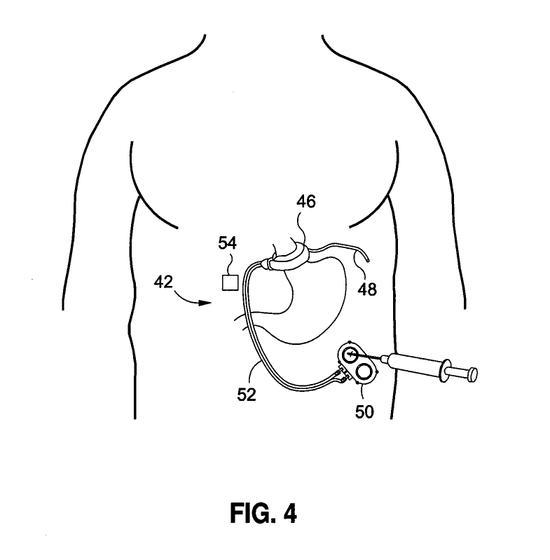

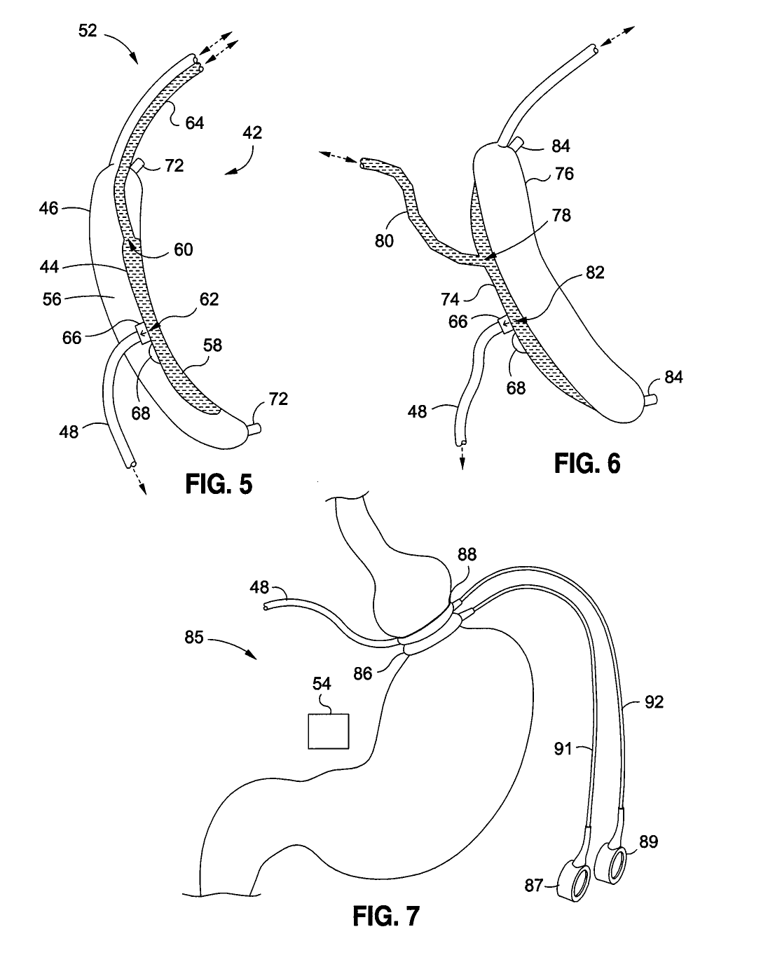

FIG. 4 illustrates an embodiment of the present invention including an implantable system 42 having a reservoir 44 (visible in FIG. 5) that is configured to contain an active agent that is distributed to a portion of the patient's body. The active agent may comprise any of the active agents discussed throughout this application, including a satiety inducing agent that is a hormone, for example a peptide hormone. The hormone may comprise at least one agent selected from a group consisting of Glucagon-like peptide (GLP-1), Oxyntomodulin (OXM), Peptide YY (PYY) and Peptide YY (3-36) (PYY (3-36)), Pancreatic Polypeptide (PP), Insulin, Leptin, Gastrin, Ghrelin blocker, inhibitors of DPP-IV, and Amylin. In addition, the satiety inducing agent may be Cholecystokinin (CCK) or Cholecystokinin 8 (CCK-8) or Pro-opiomelanocortin (POMC), or others, or any combination of the above.

The active agent may also be an agent selected from a list of agents consisting of Glial-Derived Neurotrophic Factor (GDNF); Serotonin; Dopamine and its Analogues such as: Ibogaine, Noribogaine, 18-MC, and Cabergoline; Ciliary-derived Neurotrophic Factor (CNTF); Cocaine-Amphetamine Regulated Transcript (CART); Serotonin and its Analogues; Gastric Inhibitory Peptide or Glucose-dependant Insulinotropic Peptide (GIP); Neuropeptide Y (NPY) receptor antagonists and iRNA/siRNA; Orexin A and B receptor antagonists and iRNA/siRNA; Agouti Related Peptide (AgRP) receptor antagonists and iRNA/siRNA; Cannabinoid receptor antagonists and iRNA/siRNA; the Melanocortins: Pro-Opiomelanocortin (POMC), Alpha and Beta Melanocyte Stimulating Hormone (α and β MSH); Melanin Concentrating Hormone (MCH) receptor antagonists and iRNA/siRNA; Adenosine Mono-Phosphate activated protein Kinase (AMPK); 5-aminoimidazole-4-carboxamide-1-β-D-ribofuranoside (AICAR); and Peroxisome Proliferator-Activated Receptor Delta Agonist (PPARδ-agonist), or others, or any combination of the above.

In one embodiment, the active agent may be contained within microspheres, that are held within the reservoir 44, or any other embodiment of a reservoir discussed throughout this application. The microspheres would be held in solution within the reservoir. The microspheres may be dispensed into the patient's body to release the active agent contained within the microspheres.

The implantable system 42 may be a gastric banding system, in which a gastric band 46 is used in conjunction with the reservoir 44 (visible in FIG. 5). The reservoir 44 may be incorporated with the gastric band 46, either being positioned within the gastric band 46 (as shown in FIG. 5) or fixed to an outer portion of the gastric band 46 (as shown in FIG. 6). A tube 48 may extend from the reservoir 44, having one end fixed to the reservoir 44 and another, open end positioned as desired within the patient's body. The open end may be capable of fixing to a desired portion of the patient's body through appropriate means, such as sutures, tacks, adhesive, and the like. The outlet, or open end, of the tube 48 is positioned such that the active agent dispensed from the reservoir 44 will pass through the tube 48 and contact the patient's body, eventually being absorbed into the patient's bloodstream. It is thus preferable if the open end of the tube 48 is fixed near an internal mucous membrane, or serous membrane of the patient's body. In one embodiment, the open end of the tube 48 is fixed between the outer muscle walls of the patient's body and the patient's internal organs, for absorption into the bloodstream. Other desirable locations include the peritoneum of the patient's body cavity, or elsewhere in the patient's abdominal cavity.

In one embodiment, a shunt may be utilized to allow the tube 48 to extend into a portion of the patient's upper intestines and distribute the active agent directly into the intestines. A shunt may be used to allow the tube 48 to extend into various other organs as desired, for endogenous location active agent secretion. In one embodiment, the active agent could be distributed subcutaneously, along the spinal column, or directly to the brain, in particular, the satiety centers of the brain.

The implantable system 42 may include an access port 50, which may be a dual access port (as shown in FIG. 4), and which may include a tube, or tubes 52 that lead from the access port 50 to the reservoir 44 (visible in FIG. 5) and/or the gastric band 46. One of the tubes 52 may lead to the reservoir 44, to add or remove a quantity of active agent from the reservoir 44. The other of the two tubes 52 may lead to a lumen of the gastric band 46, to add or remove fluid, such as saline, from the lumen of the gastric band 46, when the gastric band is implemented as a hydraulic band.

The implantable system 42 may further include a sensor 54 that is capable of detecting a desired biological characteristic, value, or property of the patient, which may include, for example, a hormone level of the patient. The sensor 54 may be capable of transmitting a signal to the reservoir 44, or a device associated with the reservoir 44 that causes the distribution rate of active agent from the reservoir 44 to be varied. For example, the signal may cause the reservoir 44 to dispense a portion of the active agent contained within the reservoir 44 to a portion of the patient's body, or to stop dispensing active agent from the reservoir 44, or to increase or decrease a rate that the active agent is dispensed from the reservoir 44.

FIG. 5 illustrates a schematic view of the reservoir 44 and gastric band 46 for use in the implantable system 42 shown in FIG. 4. The reservoir 44 comprises a chamber or housing capable of being filled with an active agent in fluid form. The reservoir 44 may comprise a flexible housing, to accommodate the movement of the gastric band 46 during implantation. The housing may be capable of stretching, or expanding, to accommodate fluid being delivered into the reservoir 44. The housing may also be capable of shrinking, or reducing in size, in response to fluid being removed from the reservoir 44. In one embodiment, the reservoir 44 may have a fixed size, yet may remain flexible, to accommodate the movement of the gastric band 46 during implantation.

In the embodiment shown in FIG. 5, the reservoir 44 is positioned within the inner lumen 56 of the band 46. The reservoir 44 has an outer surface 58 that is integral with an inner, stomach-contacting surface of the band 46. The reservoir 44 has a conduit 60 that allows the active agent to flow into, and out of, the reservoir 44. The conduit 60 couples to a tube 64, which may comprise one of the tubes 52 shown in FIG. 4 that is connected to the access port 50. A physician may then use the access port 50, to pass fluid through the conduit 60, to add or remove a quantity of the active agent from the reservoir 44, as desired. In one embodiment, a physician may also directly inject a syringe into the reservoir 44, to add or remove the active agent from the reservoir 44.

The reservoir 44 has an outlet 62, which allows the active agent to flow out from the reservoir 44, and into the patient's body, through the tube 48. The outlet 62 of the reservoir 44 may comprise an opening, or portal, and may include an outlet device 66, which allows, enhances, prevents, or impedes the ability of the active agent to exit from the reservoir 44. In one embodiment, an outlet device 66 may not be used, for example, in an embodiment wherein the patient or physician injects the active agent into the access port 50 and allows the active agent to directly flow into the patient's body.

The outlet device 66 may comprise a device selected from a group including a pump and a valve, or may comprise a combination of a pump and a valve. The pump and/or valve may be powered, either inductively from a remote device or through a battery (not shown) that may be charged prior to implantation of the implantable system 42, or may be charged inductively after implantation, through appropriate means. The pump may comprise a micro-pump, for example a piezoelectric pump capable of driving fluid through use of a diaphragm mechanism. In addition, the pump may comprise any other desired type of implantable pump or micro-pump, capable of providing equivalent operation. The valve may comprise a piezoelectric valve, for example a valve capable of allowing fluid to pass through the outlet 62 with a powered diaphragm mechanism. The valve may also comprise any other desired type of valve device or micro-valve capable of providing equivalent operation. Embodiments of pumps and/or valves that may be preferably utilized in the present invention are disclosed and discussed in U.S. patent application Ser. No. 12/428,311, titled “Remotely Adjustable Gastric Banding System,” filed on Apr. 22, 2009, the entire disclosure of which is incorporated herein by reference.

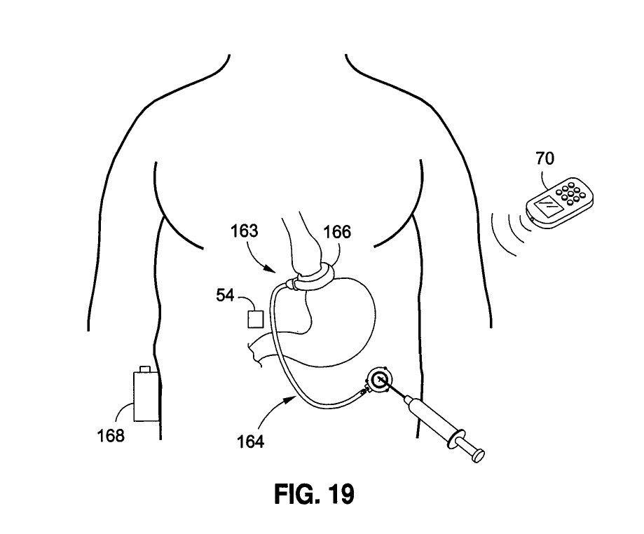

The embodiments of the outlet device 66 that include powered mechanisms (e.g., the pump and the valve) may be used in conjunction with a controller 68. The controller 68 may comprise circuitry and/or a power system capable of operating the outlet device 66 and communicating with other devices utilized in the system 42. The controller 68 may include transmitter and receiver devices, which may send and receive signals telemetrically. The controller 68 may be capable of causing the outlet device 66 to either increase a flow of active agent from the reservoir 44, or to decrease a flow of active agent from the reservoir 44, in response to signals sent by either the sensor 54 or an external controller device 70 (discussed in relation to FIG. 19). For example, the controller 68 may cause an embodiment of the outlet device 66 comprising a pump, to pump active agent from the reservoir 77 in response to a signal sent from the sensor 54. In addition, the controller 68 may be configured to open or close an embodiment of the outlet device 66 comprising a powered valve, in response to a signal sent from the sensor 54.

The receiver of the controller 68 may include an antenna, capable of receiving signals transmitted from either inside the body or outside the body. If signals are transmitted from inside the body, the signals may be sent from the sensor 54, which may be configured to wirelessly transmit signals to the controller 68. If signals are transmitted from outside the body, the signals may be sent from an external control device 70 (discussed in relation to FIG. 19). Both the sensor 54 and the external control device 70 may be capable of transmitting signals to the controller 68. The transmitted signals may cause the controller 68 to increase or decrease the rate the active agent exits the reservoir 44. For example, the transmitted signals may instruct the outlet device 66 to either pump an active agent through the outlet 62, or pump less of the active agent through the outlet 62, or to open or close a powered valve incorporated with the outlet device 66. The controller 68 may be powered by similar means as the outlet device 66, namely, through battery power or through induction.

The transmitter of the controller 68 may include an antenna, which may be the same antenna as used with the receiver, and is capable of transmitting signals outside the body. The transmitted signals may be utilized in various embodiments of the implantable system 42. For example, in one embodiment, the controller 68 may include a flow meter, and the transmitter may be capable of sending a signal to a physician, indicating whether a certain amount of active agent has passed through the outlet 62. In one embodiment, the controller 68 may detect whether the pump is operating, or the valve is open. In this embodiment, the transmitter may alert a physician when the active agent is being distributed from the reservoir 44. In one embodiment, a pressure sensor may be incorporated with the reservoir 44, capable of signaling to the controller 68 when the pressure level of the reservoir 44 is low enough to require more active agent to be inserted into the reservoir 44. The transmitter may send a signal to an external control device 70 (discussed in relation to FIG. 19), indicating a fluid level or volume of the reservoir 44 to the user of the controller device 70. In one embodiment, the controller 68 may include a processor and memory, the memory being capable of storing instructions executable by the processor. The instructions may be preprogrammed into the controller 68 prior to implantation, or may be received by the receiver of the controller 68, and set into memory by the patient or physician wirelessly, after implantation. The instructions may produce any of the actions performed by the controller 68.

The sensor 54, shown in FIG. 4, may be utilized in combination with the reservoir 44 shown in FIG. 5. The sensor 54 may comprise circuitry including a biological sensor capable of detecting a desired biological characteristic, property, or value. The biological characteristic may be a hormone level, which may be detected through means known to those skilled in the art. For example, a hormone level of the patient may be measured by measuring the dielectric constant of interstitial fluid, intra-peritoneal fluid, or blood plasma, across two electrodes, in a manner that reflects the hormone concentration of the patient. The sensor 54 may include a receiver and a transmitter, which are respectively capable of receiving and sending signals either to the controller 68 of the reservoir 44, or to a receiver located exterior to the patient's body.

The sensor 54 may be configured to cause a signal to be sent to the controller 68 or an external control device 70, in response to the measured biological characteristic of the patient. For example, the sensor 54 may be configured to store a threshold detection level for a biological characteristic within a patient's body. If the detected biological characteristic decreases below the threshold value, then the sensor may be configured to send a signal to the controller 68 of the reservoir 44 or to the external control device 70. The signal received by the controller 68 may cause the controller 68 to instruct, control, or power, the outlet device 66 to vary a rate the active agent is dispensed from the reservoir 44 and delivered to a portion of the patient's body (e.g. the part of the patient's body where the output of the tube 48 is located). In this embodiment, the signal will preferably increase the rate the active agent is dispensed from the reservoir 44. In addition, or alternatively, a threshold detection level may be stored or set in the sensor 54 that represents an upper limit of a detected biological characteristic. For example, if the sensor 54 detects the biological characteristic is above a threshold level, then the sensor 54 may send a signal to the controller 68 to instruct, control, or power, the outlet device 66 to reduce the amount of active agent being dispensed from the reservoir 44. The sensor 54 and reservoir 44 may thus act in a closed feedback loop, which allows the amount of fluid dispensed from the reservoir 44 to be controlled, at least in part, by a biological characteristic of the patient's body. If the biological characteristic is a hormone level, the sensor 54 and reservoir 44 may then act in a feedback loop to control the hormone level of the patient. In one embodiment, the sensor 54 may communicate with the controller 68 through wired means, if equivalent operation is produced. The sensor 54 may be powered, either inductively from a remote device or through a battery (not shown) that may be charged prior to implantation of the implantable system 42, or charged inductively after implantation, through appropriate means.

In one embodiment, the sensor 54 may include a processor and a memory. The processor may be capable of executing instructions stored in the memory. The instructions may be preprogrammed into the sensor 54 prior to implantation, or may be received by the sensor and set into memory by the patient or physician wirelessly, after implantation. The instructions may comprise any of the actions and responses performed by the controller 68. For example, the instructions may include the threshold detection values set to be detected by the sensor 54.

The gastric band 46 shown in FIG. 5 may comprise a hydraulic gastric band 46, having an interior lumen 56 capable of being filled with a fluid, such as saline. The gastric band 46 is configured to form a loop around a portion of the patient's stomach, creating a stoma that restricts the flow of food to the lower portion of the patient's stomach. A link device or a plurality of links 72 may connect to each other to secure the gastric band 46 in position around the patient's stomach. In addition, the links 72 may be configured to be directly coupled to a portion of the patient's stomach, through sutures or other appropriate means.

The reservoir 44 is implanted within the patient's body preferably through laparoscopic means. In other words, laparoscopic tools are used to insert the reservoir 44 into the patient's body and fix the reservoir 44 to a portion of the patient's body. If the reservoir 44 is incorporated with a gastric band 46, as shown in FIG. 5, then the same procedure used to install the gastric band 46 around a portion of the patient's stomach will be used to accordingly install the reservoir 44 around the portion of the patient's stomach. After the reservoir 44 is in position, the tube 48, leading from the outlet 62 of the reservoir may be placed within the patient's body in a desired position.

The reservoir 44 acts to dispense the active agent to the patient's body, and, as shown in FIG. 5, does so in combination with a gastric band 46 configured to restrict a portion of the patient's body. The use of the reservoir 44 thus serves to enhance the therapeutic properties of the gastric banding obesity treatment, by distributing an active agent that promotes satiety signals of the patient, or alters the metabolism of the patient, which will cause the patient to lose weight. The dosage of the active agent that is distributed by the reservoir 44 may be adjusted by a physician to accommodate various personal properties of the patient (e.g., weight loss goal, size of the patient, results of the gastric band and active agent treatment). For example, a physician may communicate with the sensor 54 to set a certain biological threshold detection level (e.g., a hormone threshold detection level), which corresponds to the personal properties of the patient. The sensor 54 may send a signal to the controller 68 or the external control device 70 if a measured biological characteristic deviates from the threshold detection level. In addition, a physician may communicate with the reservoir 44, via the controller 68, to set a degree of flow and volume from the reservoir 44, or to set a pumping or flow rate of the outlet device 66, according to the personal properties of the patient. In addition, the physician may program in the controller 68 a schedule at which the active agent is dispensed from the reservoir.

In one embodiment, the output device 66 comprises a check valve, or one-way valve, that is configured to allow an active agent to flow from the reservoir 44 in response to a force being exerted against the reservoir 44, which may be a force exerted in response to a bolus of food being passed through the esophagus, stomach, or stoma formed by the gastric band 46. The force may result from gastric peristalsis. The force may pressurize the reservoir 44 to a degree that the one-way valve opens, and allows the active agent to exit the reservoir 44. The one-way valve may thus have a set pressure threshold, at, or above which, the one-way valve opens and allows fluid to pass through. The one-way valve may then be capable of distributing the active agent during only times when the patient is eating. The active agent may then be dispensed at a time when increased satiety signals would be more useful to reduce the volume of food consumed by the patient, namely, at the eating times of the patient. In one embodiment, the one-way valve may also be a variable one-way valve, or a one-way valve that is capable of varying the pressure the valve opens in response to. The opening pressure of the one-way valve may be varied by the physician mechanically, prior to implantation of the implantable system 42, or mechanically after implantation of the implantable system 42 (e.g., when the reservoir 44 is in position). In one embodiment, the one-way valve may operate in combination with the controller 68, and a physician may communicate wirelessly with the controller 68, to adjust the threshold pressure of the one-way valve.

A possible drawback to an embodiment with a reservoir 44 positioned within a gastric band 46, as shown in FIG. 5, is that as the active agent volume decreases, the size of the reservoir 44 may decrease, which accordingly decreases the pressure exerted by the gastric band 46 against the patient's stomach. The degree of constriction the gastric band 46 exerts against the patient's stomach may decrease over time, until the reservoir 44 is refilled by the physician. FIG. 6 illustrates an embodiment of an implantable system including a reservoir 74 positioned exterior to, and adjacent to, the gastric band 76. In this embodiment, a reduced pressure of the reservoir 74 will not greatly decrease the degree of constriction applied by the gastric band 76, as the reservoir 74 is not positioned with the lumen of the gastric band 76. In this embodiment, similar to the embodiment shown in FIG. 5, the reservoir 74 has a fluid conduit 78, which serves a similar function as the fluid conduit 60 shown in FIG. 5. The fluid conduit 78 couples to a tube 80 capable of transferring the active agent to and from an access port, which may be configured similarly as the access port 50 shown in FIG. 4. The reservoir 74 may also have an outlet 82, serving a similar function as the outlet 62 shown in FIG. 5. The outlet device 66 may have similar construction and operation as discussed above in relation to FIG. 5. The gastric band 76 shown in this embodiment operates similarly as the gastric band 46 discussed in relation to FIG. 5, as it is similarly capable of constricting a portion of the patient's stomach to form a stoma. A plurality of links 84 operate similar to the links 72 discussed in relation to FIG. 5, namely, the plurality of links 84 are capable to either connecting to each other to restrict the stomach, or are capable of fixing directly to the stomach.

FIG. 7 illustrates an embodiment of an implantable system 85 that includes a reservoir 88 being separate, or not directly connected to, a gastric band 86 wrapped around a portion of the patient's stomach to form a stoma. The reservoir 88 is directly connected to the patient's stomach. The reservoir 88 connects to an access port 89 through a tube 92. The gastric band 86 connects to an access port 87 via a tube 91. The sensor 54 utilized in this embodiment may have a similar construction and operation as the sensor 54 discussed in relation to FIGS. 4 and 5.

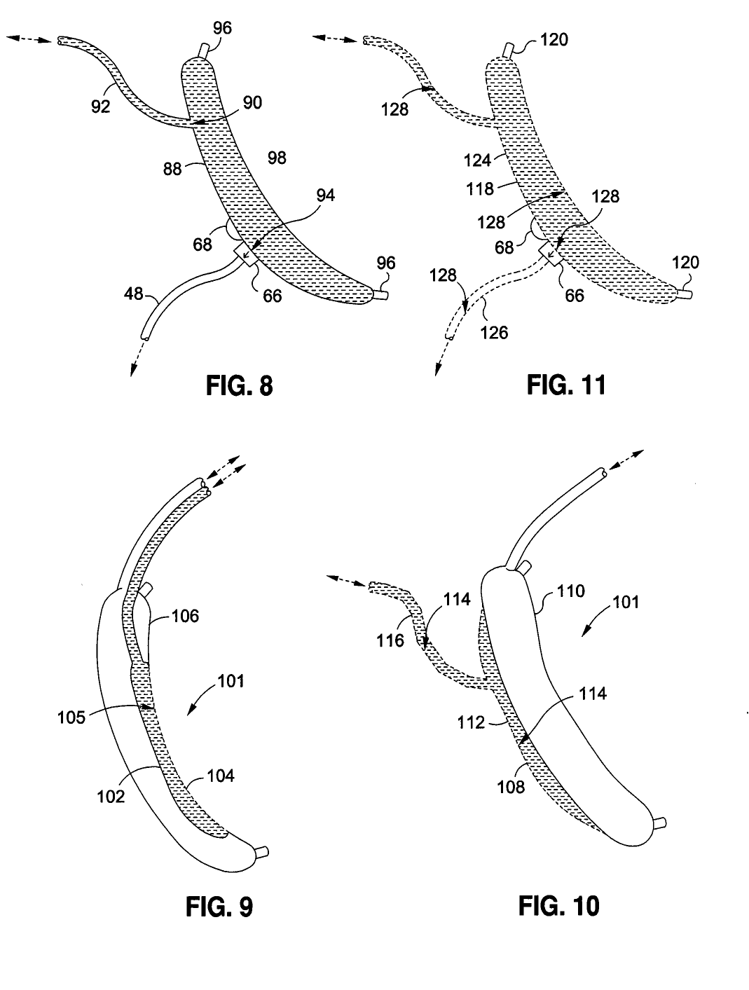

FIG. 8 illustrates the reservoir 88 shown in FIG. 7. The reservoir 88 has a similar construction and operation as the reservoirs 44, 74 shown in FIGS. 5 and 6, in that the reservoir 88 comprises a housing capable of containing a volume of active agent. The reservoir 88 in this embodiment may be made flexible, to allow the reservoir 88 to wrap around a portion of the patient's stomach during laparoscopic introduction of the reservoir 88. The reservoir 88 includes a fluid conduit 90, which has a similar construction and operation as the conduits 62, 82 shown in FIGS. 5 and 6. The fluid conduit 90 connects to the tube that leads to the access port 89 shown in FIG. 7. The reservoir 88 includes an outlet 94 in fluid communication with an outlet device 66, which has a similar construction and operation as any of the embodiments of outlet device 66 discussed in relation to FIGS. 5 and 6. The reservoir 88 includes links 96 that allow the reservoir 88 to either wrap entirely around a portion of the patient's stomach, to form a stoma, in an embodiment where the links 96 connect to each other. The reservoir 88 may therefore form an inner stomach-facing surface 98 that contacts the patient's stomach.

In one embodiment, the reservoir 88 may be configured to not wrap entirely around a portion of the patient's stomach. In this embodiment, the links 96 do not connect to each other. The links 96 may rather be connected directly to the patient's stomach. The links 96 may be tied to the patient's stomach by sutures or other equivalent attachment means.

In one embodiment, the reservoir 88 may comprise an inflexible housing that does not extend entirely around the patient's stomach. The reservoir 88 may only be tethered to a portion of the patient's stomach. The reservoir 88 may be made inflexible, as it no longer must be shaped to wrap around the patient's stomach.

In one embodiment, the reservoir 88 is not positioned around the patient's stomach. The reservoir 88 need not be positioned around a portion of the patient's stomach because the reservoir 88 is no longer joined with a gastric band, for example the gastric band 86 shown in FIG. 7. The reservoir 88 may be positioned anywhere desired within the patient's body where equivalent results are produced. For example, the reservoir 88 may be positioned along various portions of the patient's gastrointestinal tract, including on the patient's stomach, esophagus, intestines, or bowels. In addition, the reservoir 88 may be placed elsewhere within the patient's body, for example, tethered to the muscle or fat layers of the patient's body. The reservoir 88, although it may be positioned in various locations, is still desirably placed and fixed to the patient's body using laparoscopic means, to reduce the complexity of the surgical placement of the reservoir 88. Although the reservoir 88 need not be positioned to encircle the patient's stomach, in the embodiment in which the outlet device 66 is configured as a check valve or one-way valve, it is preferred that the reservoir 88 is positioned along a portion of the gastrointestinal tract where forces would be exerted during gastric activity of the patient, for example, around the stomach or esophagus of the patient. In addition, in one embodiment, the tube 48 may not be required, as the outlet of the reservoir may be positioned near the portion of the patient's body desired to receive the active agent.

FIG. 9 illustrates an embodiment of an implantable system 100 including a reservoir 102 having a semi-permeable membrane 104. The semi-permeable membrane 104 may comprise an outer surface of the reservoir 102, and, in the embodiment shown in FIG. 9, may comprise an inner, stomach-facing surface of the reservoir 102. The reservoir 102 shown in FIG. 9 may be incorporated within a gastric band 106, which may have a similar construction and operation as the gastric bands 46, 76, discussed in relation to FIGS. 5 and 6. The semi-permeable membrane 104 may comprise a silicone or nanostructure material capable of selectively diffusing the active agent through the membrane 104. The membrane serves as an outlet 105 for the reservoir 102, allowing the active agent to diffuse through the membrane 104 based on a concentration difference, or mass action, of the active agent on one side of the membrane 104, in relation to the concentration on the other side of the membrane 104. In the embodiment shown in FIG. 9, the semi-permeable membrane 104 may be positioned to directly abut, or contact the patient's stomach. The active agent therefore diffuses through the membrane 104 and be absorbed by local surfaces around the patient's stomach. Because the semi-permeable membrane 104 serves as the outlet 105 of the reservoir 102, an outlet device, for example the outlet device 66 shown in FIGS. 5, 6, and 8 is unnecessary. However, in one embodiment, the outlet device 66 may be incorporated with a gastric band having a semi-permeable membrane, to increase the flow of active agent from the reservoir, as shown, for example in FIG. 11.

The reservoir associated with the semi-permeable membrane may be positioned in various locations in the patient's body, as desired, and the semi-permeable membrane may form various portions and surfaces of an implantable system, as desired. For example, FIG. 10 illustrates an implantable system 101 including a reservoir 108 positioned outside of a gastric band 110. The gastric band 110 may have a similar construction and operation as the gastric band 76 shown in FIG. 6. The reservoir 108 may include a semi-permeable membrane 112 that operates similarly as the membrane 104 shown in FIG. 9. The semi-permeable membrane 112 serves as the outlet 114 for the reservoir 108. In this embodiment, a portion of the tube 116, or a portion of the access port, for example the access port 50 shown in FIG. 4, may be made from a semi-permeable membrane, capable of distributing the active agent to the patient's body.

FIG. 11 illustrates an embodiment of an implantable system including a reservoir 118 that is not directly connected to a gastric band. The reservoir 118 includes links 120 that have a similar construction and operation as the links 96 shown in FIG. 8. The links 120 allow the reservoir 118 to either wrap entirely around a portion of the patient's stomach, to form a stoma, in an embodiment where the links 120 connect to each other. In addition, the links 120 may be directly fixed to the patient's stomach, in an embodiment where the reservoir 118 does not wrap entirely around a portion of the patient's stomach. The reservoir 118 includes a semi-permeable membrane 124 that has a similar construction and operation as the membranes 104, 112 discussed in relation to FIGS. 9 and 10. The reservoir 118 shown in FIG. 11 may be positioned similarly as the reservoir 88 shown in FIG. 8, namely, the reservoir 118 may be positioned anywhere within the patient's body or along the gastrointestinal tract, as desired. For example, the reservoir 118 may be positioned such that the semi-permeable membrane 124 contacts and diffuses the active agent to a highly vascularized and permeable tissue such as a mucous or serous membrane within the patient's body.

The reservoir 118 may additionally utilize the various embodiments of the outlet device 66 and the controller 68, which may operate in conjunction with the sensor 54, as discussed in various other embodiments described in this application. The tube 126 leading from the outlet device 66 may be made of a semi-permeable membrane. The semi-permeable membrane 124 may comprise the outlet 128 of the reservoir 118, in combination with the outlet 128 that is coupled to the outlet device 66.

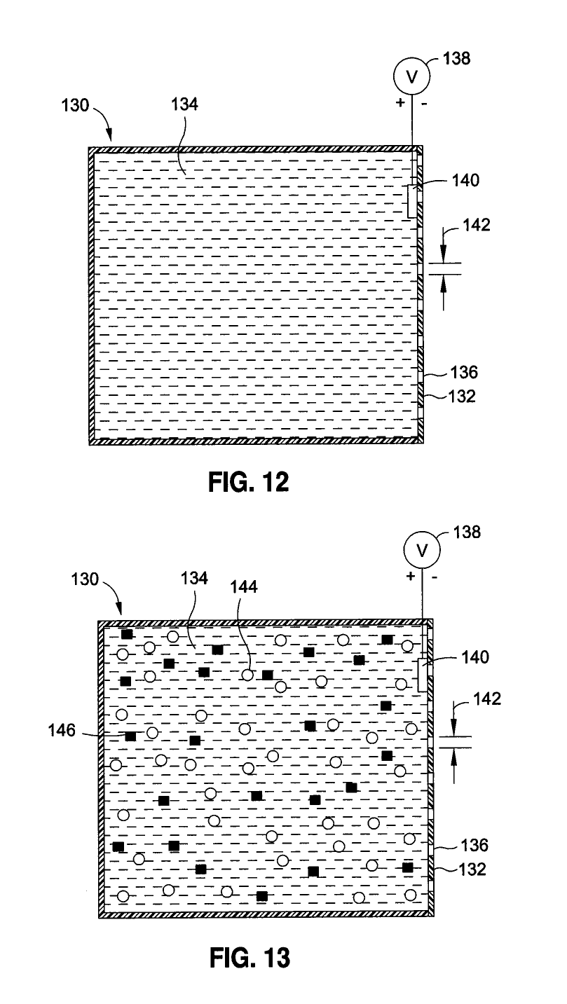

FIG. 12 illustrates a schematic representation of a reservoir 130 having a semi-permeable membrane 132. The reservoir 130 may represent any of the reservoirs discussed in relation to FIGS. 9-11, or any portion of an implantable system having a semi-permeable membrane, discussed in relation to FIGS. 9-11. The reservoir 130 includes a central chamber or housing, configured to contain an active agent 134, represented by dashed lines in FIG. 12. An outer surface of the reservoir 130 comprises a semi-permeable membrane 132, having a series of pores 136 for the active agent 134 to pass through and exit the reservoir 130. The active agent 134 may diffuse through the semi-permeable membrane 132 based on a diffusive force, caused by a concentration differential of the active agent 134 on one side of the membrane 132 in relation to the other side of the membrane 132. The rate of diffusion may depend in part on the porosity of the membrane 132, the diffusibility of the active agent across the surface of the membrane 132, and the surface area to volume ratio of the reservoir 130. Such design features of the reservoir 130 may be varied to produce a desired effect.

In one embodiment, the passage of the active agent 134 from one side of the membrane to the other may be aided or hindered by use of a voltage source 138 and an electrode 140, utilized in combination with the reservoir 130. The voltage source 138 may be powered, either inductively from a remote device or through a battery (not shown) that may be charged prior to implantation of the implantable system or charged inductively after implantation, through appropriate means. The electrode 140 may be positioned on either side of the membrane 132, or on both sides of the membrane 132, or generally within the reservoir 130 as desired. The voltage source 138 and the electrode 140 operate to form an electric charge on one side of the membrane 132, or on both sides of the membrane 132. The charge may enhance or impede the diffusion of the active agent from the reservoir 130 to the patient's body, as the active agent 134 may comprise molecules having a net charge, or a polarity. Based on the charge of the molecules forming the active agent, the electrode 140 may cause a charge to be formed on either side of the membrane 132 that either draws the active agent 134 out from the reservoir, or serves to keep the active agent 134 within the reservoir 130, through an electric force.

The desired polarity of the charge formed on either side of the membrane 132 depends on the polarity of the molecules that were selected to comprise the active agent 134. In one embodiment, the net charge formed by the electrode 140 on either side of the membrane 132 may be varied as desired by the voltage source 138. In this embodiment, the voltage source 138 may be a variable voltage source. The voltage output by the voltage source 138, and the polarity of the net charge formed by the voltage source 138, may be controlled by, for example, the controller 68 discussed in relation to FIGS. 5, 6, 8 and 11. The controller 68 may be appropriately configured to instruct the voltage source 138 to produce a charge in response to a signal from the sensor 54, or the external control device 70 (shown in FIG. 19). For example, the controller 68 may receive a signal from the sensor 54, directing the voltage source 138 to increase or decrease the strength, or polarity, of the net charge formed on either side of the membrane 132. If the sensor 54 detects a low hormone level in the patient, the sensor 54 may send a signal to the controller 68 instructing the controller 68 to increase the flow of the active agent 134 through the membrane 132. The controller 68 may instruct the voltage source 138 to increase the net charge, or vary the polarity of the charge, on one side of the membrane 132, if such an action will increase the flow of the active agent 134 through the membrane 132. A signal received from the external control device 70 may produce a similar result.

In one embodiment, the controller 68 may be configured to control the voltage source 138 to increase or decrease the strength or polarity of the net charge formed on either side of the membrane 132, according to a schedule. The controller 68 may be pre-programmed with a control schedule that defines the times the voltage source 138 enhances or impedes diffusion of the active agent 134 through the membrane 132. The control schedule may be configured to enhance or impede the diffusion of the active agent 134 through the membrane 132 at specified times during the day. For example, the control schedule may include a schedule of meal times. The amount of active agent 134 dispensed may increase at the defined meal times. In addition, the control schedule may be set to reduce the flow of the active agent 134 from the reservoir 130 during times when food consumption is not likely, for example, during times when the patient is likely to be sleeping. The control schedule may be configured to be varied or reprogrammed telemetrically after the controller 68 has been implanted. The control device 70, discussed in relation to FIG. 19, may be used to reprogram the controller 68.

In one embodiment, a pressure sensor may be incorporated with the reservoir 130. The pressure sensor may be capable of detecting a force exerted against the reservoir 130 in response to a bolus of food being passed through the patient's esophagus or stomach. The force may result from gastric peristalsis. The pressure sensor may send a signal to the voltage source 138 to increase the flow of the active agent 134 through the membrane 132 in response to the force applied to the reservoir 130.

The response of the voltage source 138 to a signal from the sensor 54, or the external control device 70, or pressure sensor, may be varied as desired (e.g., the voltage source 138 may decrease the net charge on one side of the membrane 132, if doing so would increase flow of the active agent 134 through the membrane 132). In one embodiment, the electrode 140 may equivalently comprise a plurality of electrodes, positioned throughout, and/or external to the reservoir 130, if equivalent operation results.

In one embodiment, the voltage source 138 and the electrode 140 may be utilized to vary the size 142 of the pores 136 of the membrane 132. The membrane 132 may be made of a material structured to contract or expand in response to an electric voltage applied to the membrane 132. The material of the membrane 132 is composed such that the pore size 142 depends on the voltage being applied to the membrane 132, for example, the material may comprise an electroactive polymer having a series of pores. The size 142 of the pores 136 depends on the presence of a voltage applied by the electrode 140, which may either increase or decrease the size 142 of the pores 136. The active agent 134 may either more easily or less easily flow, through the membrane 132, if a voltage is applied to the membrane 132 by the electrode 140. In one embodiment, the voltage source 138 may be a variable voltage source 138, and may be controlled by the controller 68 discussed in relation to FIGS. 5, 6, 8 and 11. The controller 68 may operate in response to a signal produced by the sensor 54 or an external control device 70 (shown in FIG. 19). For example, if the sensor 54 detects a low hormone level in the patient, the voltage source 138 may appropriately increase or decrease the voltage applied to the membrane 132, to increase the pore size 142, and increase the flow of the active agent 134 from the reservoir 130.

In one embodiment, the semi-permeable membrane 132 may be configured to vary the pore size 142 without the use of the voltage source 138 and electrode 140. In this embodiment, the semi-permeable membrane 132 may be made from a material that varies in pore size 142 automatically in response to an environmental condition, based on the material properties of the membrane 132. The material may comprise polyethylene, or polyethylene filled with SiO₂. Such desirable materials are discussed in the publication, “Response of Filled Polyethylene Membranes to the Changes in the Environmental Conditions,” M. A. Islam and N. D. Nikolov, volume forty-five, issue 8, Journal of Applied Polymer Science, the entirety of which is incorporated by reference. The environmental condition may comprise a biological property, for example, a pH level, a temperature, or an internal pressure of the patient. The material of the membrane 132 may be selected such that the pore size 142 automatically adjusts in response to the environmental condition, and will adjust to a desired degree in response to the environmental condition. Thus, for example, if a pH level in a certain level is present within a response range of the membrane 132, the pore size 142 may increase or decrease to vary the flow of active agent 134, as desired.

FIG. 13 illustrates an embodiment of the reservoir 130 shown in FIG. 12, which utilizes biological organisms 144 to produce the active agent 134. The biological organisms 144 are represented in FIG. 13 as circles, and may comprise any biological organism, preferably at the size of a microbe, or as would otherwise fit into a reservoir 130 for implantation within a patient's body. The organisms 144 may preferably comprise any cell or combination of zoograph cells, autograph cells, autograph cells, bacterium, algae, or yeast. The organisms 144 are stored within the reservoir 130 and are sustained by a nutrient media 146, represented in FIG. 13 as squares. The reservoir 130 is configured in a manner to properly store and keep the organisms 144 alive for an extended period of time. For example, the pores 136 are sized such that the organisms 144 cannot pass through the membrane 132, yet the active agent 134 produced by the organisms 144 may still pass through the pores 136. The pores 136 are preferably sized to be as small as possible while still allowing the diffusion of the agent across the membrane 132. Enough nutrient media 146 is supplied to the reservoir 130 to sustain the organisms 144 for an extended period of time. The internal volume of the reservoir 130 and nature of the nutrient media 146 selected should optimize the longevity, reproductive capabilities, health, and number of organisms 144. The reservoir 130 is designed to be safe from rupture. In one embodiment, the organisms 144 may be contained within microspheres to more effectively protect the organisms. The microspheres may be placed directly into the patient's body, without the use of the reservoir 130. In one embodiment, the microspheres may be injected intravenously into the patient's body.

The organisms 144 may be preferably sustained within the reservoir 130 for a period of no less than six months. A lifetime of up to thirty years may be reached. The sustainable duration of the organisms 144 may be varied, based on the scope of treatment desired by the patient, or the severity of the patient's obesity problem. The reservoir 130 may otherwise be configured in any manner discussed in reference to FIG. 12, including an embodiment in which a net charge is produced on a side of the membrane 132 to enhance or impede flow of the active agent, or an embodiment in which the size of the pores 136 is variable due to an applied voltage or environmental factors. The organisms 144, active agent 134, and nutrient media 146 may be replenished by any method discussed throughout this application, including replenishment through an access port.

The organisms 144 utilized in the reservoir 130 may comprise bacteria, due to the relatively non-complex structure of bacterial DNA. The bacteria may be engineered to produce a desired active agent 134 as a result of biological engineering of the bacteria's DNA. Prior to the bacteria being implanted in the reservoir 130, a plasmid vector may be introduced into the bacteria containing a strand of DNA that will cause the bacteria to produce the desired active agent 134. The plasmid vector may be formed by splicing a desired sequence of DNA into the plasmid vector. The DNA sequence may be capable of producing the agent of interest, for example, the CCK-8 (cholecystokinin octapeptide) sequence may be used if desired. The DNA sequence is spliced into a vector having the appropriate promoter section. The vector may comprise a bacteriophage vector, a arbovirus vector, a lentiviral vector, a plasmid vector, a herpes simplex viral vector, a semliki forest viral vector, a vesicular stomatitis viral vector, a baculoviral vector, an autographa californica nuclear polyhedrosis viral vector, and a ribonucleic acid interference (RNAi) via small interfering ribonucleic acids (siRNA). Other vectors may be utilized as desired, to produce an equivalent result. Once the vector has been introduced to the bacteria, the bacteria act as a self-replicating carrier of a vector which codes for the desired active agent. After the vector has been incorporated into the bacterial DNA, the bacteria containing the modified, or chimeric, strand of DNA will be replicated, amplified and reproduced. Methods of producing recombinant DNA are discussed in “AN INTRODUCTION TO GENETIC ANALYSIS” by Anthony Griffiths, Jeffery Miller, David Suzuki, Richard Lewontin, and William Gelbert. Further information by also be found in “MOLECULAR CELL BIOLOGY” by Harvey Lodish, Arnold Berk, Paul Matsudaira, Chris Kaiser, Monty Krieger, Matthew Scott, S. Lawrence Zipursky, and James Darnell. In one embodiment, electrical energy may be applied to the bacteria to weaken the cell wall of the bacteria. A plasmid is then introduced into the bacterial cell, having a DNA sequence that codes for a desired active agent. The bacterium will express the genes in the plasmid and produce the desired active agent.

Once a stable colony of chimeric bacteria are produced, the bacteria will then be introduced into the reservoir 130, either prior to implantation of the reservoir 130 within the patient's body, or after the reservoir 130 has been implanted. The bacteria may be introduced into the reservoir 130 by either direct introduction of the bacteria into the reservoir 130 (e.g., direct injection by a syringe), or through an access port, for example, the access port 89 shown in FIG. 7. The active agent produced by the bacteria is selected such that no significant adverse symptoms are produced for the patient. The active agent is introduced to the patient at a rate sufficient to produce the desired treatment effect. In an embodiment wherein cholecystokinin octapeptide is utilized as the active agent, the desired rate of diffusion into the patient's body may be 1.6 picomole per liter per minute. Such a diffusion rate may produce a desired concentration of cholecystokinin octapeptide within the patient's body of 4+/−0.5 picomoles per liter when the patient is fasted, and 8+/−1.5 picomoles per liter when the patient is fed. In an embodiment wherein peptide-tyrosine-tyrosine (3-36) (PYY 3-36) is utilized as the active agent, the desired rate of diffusion into the patient's body may be 0.3182 picomole per liter per minute. Such a diffusion rate may produce a desired concentration of peptide-tyrosine-tyrosine (3-36) within the patient's body of 11+/−1 picomoles per liter when the patient is fasted, and 20+/−1 picomoles per liter when the patient is fed. Such diffusion rates and body concentrations are approximate, and may be varied as desired.

A benefit of utilizing biological organisms 144 to produce the active agent 134 is that the active agent may be replenished within the patient's body over an extended duration of time. A quantity of new active agent 134 is produced by the organisms 144 during the treatment period of the patient, rather than a single quantity of active agent 134 being inserted into the reservoir 130 by a physician and then remaining in the patient's body, and being exposed to the internal body heat of the patient, for an extended duration of time. The biological organisms 144 may extend the effective life of the active agent 134 dispensed into the patient's body. In addition, the organisms 144 may reduce the number of times the reservoir 130 must be refilled. It may be beneficial to reduce the number of refills of the reservoir 130, if an access port is used to fill the reservoir 130. The physician would not need to insert a needle into the patient's body as often, which would reduce pain felt by the patient over the course of treatment.

The embodiments shown in FIGS. 9-13 that utilize a semi-permeable membrane may be incorporated with any of the embodiments shown throughout this application. For example, the semi-permeable membrane may be utilized with any part of other implanted components of the system 10 shown in FIGS. 1-3, for example, the fluid line 16 and/or access port 18. The semi-permeable membrane may allow for a slow, for example, constant, diffusion of the agent into the body from other locations in the body.

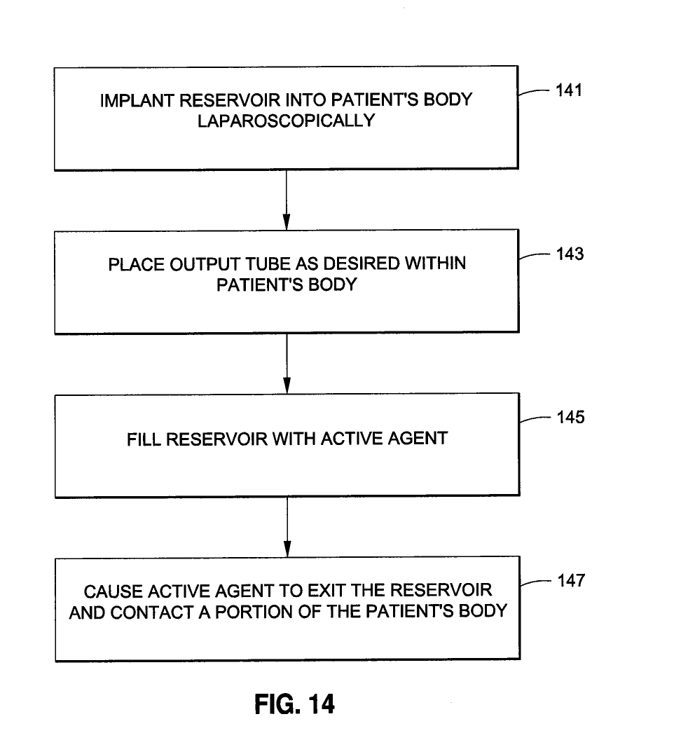

FIG. 14 illustrates an exemplary method for the treatment of obesity utilizing any of the reservoir or gastric banding embodiments shown in FIGS. 4-13. In step 141, a reservoir is implanted within the patient's body laparoscopically, or through laparoscopic means. Laparoscopic tools are utilized to insert the reservoir into the patient's body. The reservoir may be incorporated with a gastric band, and may comprise any of the reservoir embodiments shown in FIGS. 4-13. The step of implanting the reservoir into the patient's body laparoscopically may include fixing the reservoir to the desired portion of the patient's body. The reservoir may be fixed to any portion of the patient's body discussed throughout this application as being desired, including along the patient's gastrointestinal tract, to the patient's muscle wall, or around a portion of the patient's stomach. The reservoir may be wrapped around a portion of the patient's stomach to form a stoma. If the reservoir is incorporated with a gastric band, the reservoir will be fixed to the portion of the patient's stomach that the gastric band wraps around.

In step 143, an output tube, or tube 48, 126 shown in FIGS. 4-8, and 11, may be placed as desired within the patient's body. The tube may be placed using laparoscopic tools. The tube is preferably fixed to a portion of the patient's body that allows the active agent to be dispensed to mucous or serous membranes within the patient's body. The tube may dispense the active agent to any other desired location discussed throughout this application. The tube may be fixed to any desired portion of the patient's body discussed throughout this application, including a muscle wall. The tube may be fixed to a portion of the patient's body with sutures, tacks, an adhesive, or the like.

In step 145, the reservoir is filled with the active agent. The reservoir may be filled through an access port, or through direct injection of the active agent into the reservoir. In one embodiment, the reservoir may be pre-filled with an active agent prior to implantation. For the embodiment shown in FIG. 13, the filling step may further comprise placing organisms and/or nutrient media into the reservoir.

In step 147, the physician or patient may cause the active agent to be dispensed from the reservoir, through any of the means discussed throughout this application. For example, the physician may activate a pump to dispense the active agent from the reservoir. In addition, the physician may implant and activate a biological sensor, capable of acting in a feedback loop with the reservoir, as discussed in relation to FIG. 5. The active agent is dispensed to contact the desired portion of the patient's body.

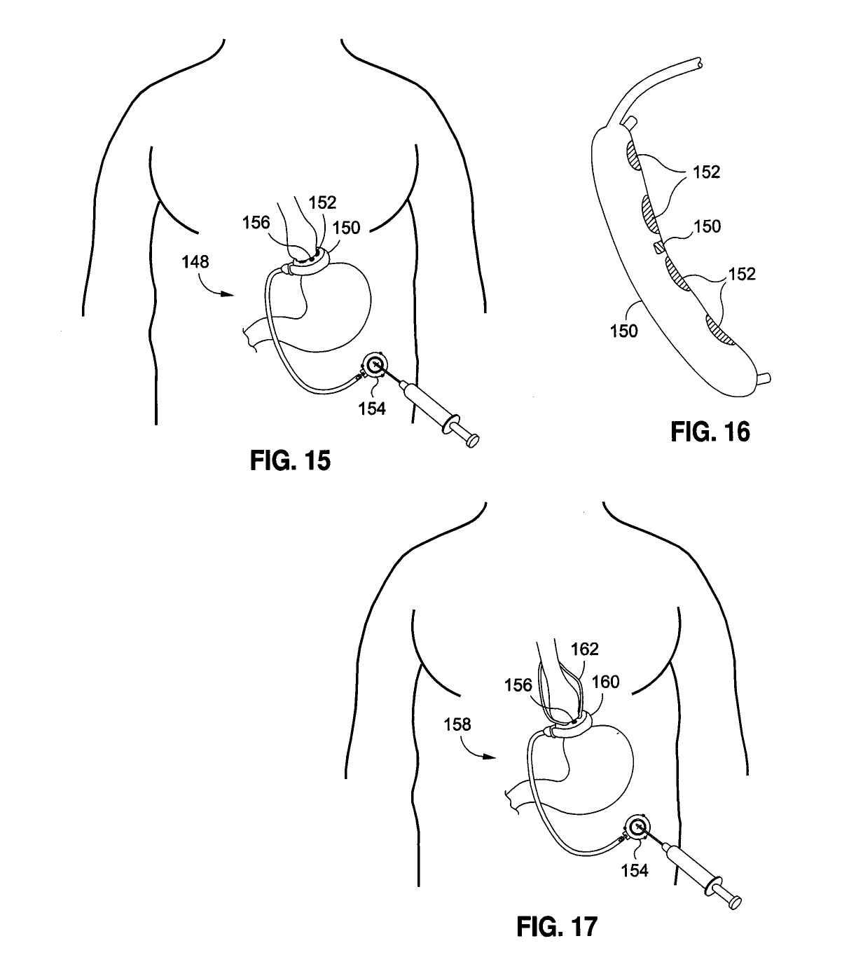

FIG. 15 illustrates an embodiment of an implantable system 148 including a gastric band 150 having a plurality of electrodes 152 configured to apply electrical energy, or stimulation, to the portion of the patient's stomach being constricted. The gastric band 150 may comprise a hydraulic gastric band, in fluid communication with an access port 154 used to fill the band 150. The implantable system 148 may further include an electrical control device 156 capable of containing instructions in a memory, which are executed by a processor. The electrical control device 156 may also include a transmitter and receiver, the receiver being capable of receiving instructions from an external transmitter sent wirelessly, to allow the electrical control device 156 to be programmed after implantation. The electrical control device 156 may also be programmable prior to implantation. The instructions stored in the memory may cause the electrodes 152 to apply electrical energy to the patient's stomach in response to a force exerted by the patient's gastric tract, or on a timer or schedule stored in the memory.

The electrical control device 156 may additionally include a pressure sensor, capable of sensing gastric activity of the patient. For example, the sensor may detect a bolus of food passing through the gastro-intestinal tract of the patient. The electrical control device 156 may then instruct the electrodes 152 to apply electrical energy to the patient's stomach, in response to the force detected by the control device 156. The electrical control device 156 may be powered, either inductively from a remote device or through a battery (not shown) that may be charged prior to implantation of the implantable system 148 or charged inductively after implantation, through appropriate means.

The application of electrical energy to the patient's stomach, used in conjunction with a gastric band 150, may serve to promote satiety signals delivered to the patient's brain. The electrical energy may stimulate local nerves that are normally only stimulated when food passes to the patient's stomach. The electrical impulses may strengthen the intensity of these signals to indicate to the patient's brain that more extensive food consumption has occurred than has actually occurred. The patient may then feel full more quickly, and will reduce food consumption sooner. The use of a pressure sensor in the electrical control device 156 may assure that the enhanced satiety signals are produced when most relevant for the patient, namely, during times of food consumption.

FIG. 16 illustrates the gastric band 150 shown in FIG. 15. The electrodes 152 are positioned along an inner stomach-facing surface of the band 150, to apply electrical energy to the local tissues of the stomach. The electrical control device 156 is positioned to sense a force exerted against the band 150 in response to gastric activity.

FIG. 17 illustrates an embodiment of an implantable system 158 including a gastric band 160 having a plurality of electrodes 162 that couple to the lower third of the patient's esophagus. The electrodes 162 extend from the gastric band 160, and may be powered, and controlled by the electrical control device 156, in the same manner as the electrodes 152 discussed in relation to FIGS. 15 and 16. For example, the electrodes 162 may be configured to apply electrical energy to the lower third of the patient's esophagus in response to a force detected by the electrical control device 156, and exerted by the patient's gastro-intestinal tract. The electrodes 162 are fixed to the patient's esophagus by known means, including an adhesive, barbs, sutures, or similar other means. The electrodes may comprise thin, wire-like projections extending from the gastric band 160 in a direction away from the patient's stomach.

A benefit of placing an electrode 162 along the lower third of the esophagus, or lower thoracic esophagus, is to enhance the effect of the electrical stimulation applied to the patient's body. Recent studies suggest direct stimulation to the lower third of the esophagus may produce enhanced stimulation of local nerves, including the vagus nerve, which will enhance the production of satiety signals. The stimulation of the lower third of the esophagus, as opposed to direct simulation of the vagus nerve along other portions of the patient's body, for example, the patient's stomach, offers an improvement over prior known electric stimulation methods. In addition, the use of electrodes along the lower third of the esophagus, in combination with a gastric band positioned around a portion of the patient's stomach to form a stoma, produces a superior combination of obesity treatments over electrical stimulation alone, or gastric banding treatment alone, or electric stimulation of other portions of the vagus nerve, including along the stomach. In one embodiment, the electrode 162 may be placed along the vagus nerve in a position not along the lower third of the esophagus. However, it is understood the position of the electrode 162 along these other portions of the vagus nerve may not include the therapeutic effects of the electrodes 162 placed along the lower third of the esophagus.

In one embodiment, the direct stimulation of the lower third of the patient's esophagus does not utilize the gastric band 160, but may include a separate electrical control device configured to power and operate the electrodes 162. In one exemplary method of operation, an electrode is inserted laparoscopically within the patient's body. The electrode is then coupled to the lower third of the patient's esophagus, to offer superior production of satiety signals in response to electrical stimulation. The electrode may be powered by an electrical control device, which may be incorporated with a gastric band, or may comprise a separate device. The electrical control device may include a pressure sensor, which causes the electrodes to apply electrical energy to the patient's esophagus in response to a force exerted against the band by the gastrointestinal tract.

Any of the embodiments shown in FIGS. 15-17 may be incorporated with any other embodiment shown throughout this application. For example, electric stimulation in combination with the use of a reservoir of active agent may serve to greatly enhance the production of satiety signals produced in an individual's body. In addition, the ancillary devices 22, 122, 222, 322 may be incorporated with any of the structures used to form the electrical stimulation embodiments shown in FIG. 15-17. Any combination of treatments may be used as desired to enhance the treatment of obesity.

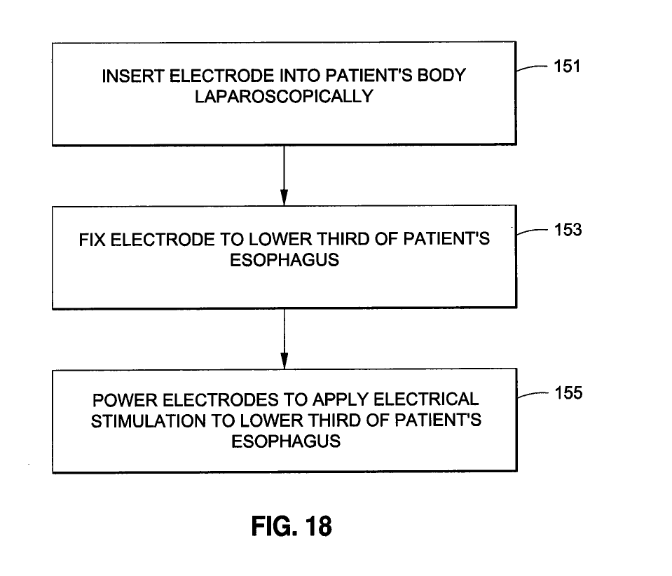

FIG. 18 illustrates an exemplary method for the treatment of obesity utilizing the electrical stimulation device embodiment shown in FIG. 17. In step 151, an electrode is inserted within a patient's body laparoscopically, or through laparoscopic means. Laparoscopic tools are utilized to insert the electrode into the patient's body. The electrode may be coupled to a gastric band, as shown in FIG. 17. A combination of gastric band treatment and electric treatment to the lower third of the patient's esophagus offers superior therapeutic properties than a gastric band used alone, or in combination with electric treatment to other portions of the patient's body.

In step 153, the electrode is fixed to the lower third of the patient's esophagus. The electrode may be fixed to the patient's esophagus using barbs, tacks, sutures, adhesives, and the like. If the electrode is coupled to a gastric band, the gastric band may be positioned around a portion of the patient's stomach, preferably the cardia, prior to, at, or after, this step. The electrode may comprise a wire-like projection extending from the gastric band and connecting to the esophagus. The electrode may be positioned along the portion of the patient's vagus nerve extending along the lower third of the esophagus. In an embodiment including a plurality of electrodes, each electrode may be positioned in sequence along the vagus nerve, or other portions of the lower third of the esophagus. In an embodiment including an electrical control device, for example the device 156 shown in FIG. 17, the electrical control device may be inserted into the patient's body prior to, at, or after, this step. The electrical control device causes the electrode to apply electric stimulation to the lower third of the patient's esophagus.

In step 155, the electrode is powered to apply electrical stimulation to the lower third of the patient's esophagus. Such power may be delivered via a battery charge, or an inductive charge. The electrical control device may be powered at this step to allow the electrical control device to cause the electrode to deliver electric stimulation to the lower third of the esophagus.

FIG. 19 illustrates an embodiment of a system 163 used for the treatment of obesity. The system 163 includes the sensor 54 and an external control device 70, which may be operated by the patient or by a physician, each equivalently referred to as the user in this application. The system 163 preferably includes a gastric banding system 164, which includes a gastric band 166 placed around the patient's stomach. The use of a gastric band 166 in combination with the therapeutic actions discussed in relation to the system 163 (e.g., application of a patch, drinking of a liquid) is designed to treat obesity in the patient to a greater degree than a treatment solely involving a gastric band.

The external control device 70 may comprise a handheld device that may be carried by the patient, or may be used by the physician. The external control device 70 may also comprise any other electrical device used external to the patient, and capable of receiving and/or transmitting information to the sensor 54. The external control device 70 may include a transmitter, a receiver, a processor and a memory. The external control device may additionally include an alerting system, which may comprise an auditory alarm or notification, or a visual stimulation or notification, such as a light or a reading on a display screen, or a physical alerting system, including movement of the external control device 70, such as a vibration. The memory may store instructions, executed by the processor. The instructions may cause the control device 70 to perform any of the operations discussed throughout this application. The external control device 70 may also include input means, such as a keypad, for the user to input instructions into the control device 70.

The receiver of the control device 70 may include an antenna capable of receiving signals sent from the sensor 54. The signal may provide information to a user, informing the user about the readings of the sensor 54. For example, the control device 70 may alert the user to take action in response to the signal sent from the sensor 54. The signal transmitted from the sensor 54 may indicate to the external control device 70 that a biological characteristic, such as a hormone level, is below a threshold value for the patient. The external control device 70 may then provide a notification, or publish certain responses to the user, for the user to take action, in response to the biological characteristic sensed by the sensor. The action preferably is effective to vary the biological characteristic sensed by the sensor. The notification, or publication, may utilize the alerting system, which may involve the sounding of an alarm, or a message presented on a display for the user to take action.

In response to the alert from the control device 70, the user may take a series of actions. Generally, the actions are designed to respond to the alert provided by the control device 70. For example, if the alert indicates a low hormone level in the patient, then the user may perform such actions that will increase the hormone level.

One such action may include injecting the patient with an active agent, possibly with a syringe. The injection may be a manual injection directly into the body of the patient. The control device 70 may alert the patient to inject the patient's body with a syringe during routine times. The injections may be performed under physician or patient control. The injections could occur routinely, or as advised by the control device 70.

Another action may include increasing the hormone level of the patient through a patch placed on the skin of the patient. The patch may have an active agent on one side of the patch, and may be capable of slowly diffusing the active agent through the patient's skin. The patch could be replaced routinely, or as advised by the control device 70.

Another action may include the patient inhaling an active agent, either through the nose or mouth or spraying an active agent into the nose or mouth. A nasal spray may allow the vaporous active agent to be applied to the nasal canal for immediate absorption. The active agent would be received by the patient closer to the satiety centers of the brain. An inhalant would allow the vaporous active agent to be absorbed into the lungs. The inhalant or spray could be administered routinely, or as advised by the control device 70.

Another action may include the patient drinking a liquid including an active agent. The active agent may be absorbed in the mouth, esophagus, or further down the gastrointestinal tract. A liquid may also be sprayed into the patient's mouth. The liquid could be administered routinely, or as advised by the control device 70.

Another action may include the patient swallowing a pill containing a desired active agent. In one embodiment, the pill may be coated to allow for slow, continuous or timed release. In one embodiment, the pill may be coated to react when in combination with a certain pH to allow it to pass into a specific location of the gastrointestinal tract. In one embodiment, the pill may have multiple mini-spheres of active agent coated with a variety of coatings controlled by pH to allow for the active agent to be released throughout the gastrointestinal tract. The pill could be administered routinely, or as advised by the control device 70.

Another action may include the user placing an orally received substance within the patient's mouth, including a film or gum that may introduce an active agent into the patient's body. The substance may be placed under the patient's tongue, and introduced into the patient's body through the local mucous membranes beneath the tongue. A chewing gum may allow the active agent to be absorbed by the mucous membranes within the patient's mouth. The orally received substance could be administered routinely, or as advised by the control device 70.

The actions, performed in combination with a gastric band therapy, will provide for a superior treatment of obesity during the duration of the treatment, in contrast to a gastric band treatment alone, or the actions performed alone.

The external control device 70 may be configured to select an appropriate alert in response to the signal sent from the sensor 54. For example, the control device 70 may be programmed to determine if the sensor 54 has indicated a hormone level is too high, and will alert the user to reduce hormone intake. In addition, the control device 70 may be programmed to determine if the sensor 54 has indicated a hormone level is too low, and will alert the user to increase hormone intake, in any form. The control device 70 may be configured to program the threshold detection level into the sensor 54, as discussed in relation to FIG. 5.

In one embodiment, the control device 70 may be configured to instruct the user on which particular action to take, based on the readings of the sensor 54. For example, the control device 70 may select whether the patient should ingest a pill having a hormone, or chew a gum having a hormone, based on the signal sent from the control device 70. The particular action selected may be based on the degree to which the biological characteristic deviates from a threshold value, or based on a schedule of therapy designed for the patient, by, for example, the physician.

In one embodiment, the sensor 54 may be incorporated with a gastric band, to provide local readings of a desired biological characteristic for the patient. The sensor 54 may telemetrically send signals to the control device 70 using a transmitter integrated with the sensor 54.

The external control device 70 may be used with various other embodiments of systems for the treatment of obesity discussed throughout this application. For example, the transmitter of the external control device may be used to transmit signals to the controller 68, discussed in relation to FIGS. 5, 6, 8 and 11. The external control device 70 may be capable of causing the output device 66 to emit, or not emit, the active agent into the patient's body, in response to instructions sent from the external control device 70. The external control device 70 may be capable of setting the rate at which the output device 66 dispenses the active agent from the reservoir. The external control device 70 may be capable of programming an active agent dispersion schedule into the controller 68. In addition, the sensor 54, the external control device 70, and the controller 68, may act in a closed loop, wherein the sensor 54 senses a biological parameter of the patient, and sends a signal to the external control device 70 that indicates to the user to take a specified action. The user may then instruct the controller 68 to distribute the active agent from the reservoir. In addition, the sensor 54 may cause the controller 68 to distribute the active agent from the reservoir without the intervention of the user. The external control device 70, however, may receive notification that the controller 68 is automatically distributing the active agent in response to the sensed biological parameter. The external control device 70 may give the user the opportunity to intervene, and prevent the automatic distribution of the active agent, or enhance the distribution of the active agent.

In one embodiment, the system 163 includes an external distribution device 168 that may be positioned external to the patient's body. The distribution device 168 may comprise a reservoir for holding an active agent that is capable of being injected into the patient's body, or pumped into the patient's body through a tube. The distribution device 168 may be capable of accurately metering the volume and rate at which the active agent is injected into the body. The external distribution device 168 may receive signals directly from the sensor 54 instructing the device 168 to distribute the active agent to the patient's body. The external distribution device 168 may automatically distribute the active agent to the body in response to this signal, or may produce an alert to the user instructing the user to take action. The user may cause the external distribution device 168 to dispense the active agent to the patient's body in response to the signal from the sensor 54. A benefit of the external distribution device 168 is that the active agent may be refilled by the patient, or physician, without having to insert a needle into an access port, or directly into a reservoir. The patient could load and self-administrate the external distribution device 168.

In one embodiment, the actions taken in response to the alert from the control device 70 may be performed solely, or without prompting from the control device 70. For example, the patient may undergo an obesity treatment including the placement of a gastric band on the patient's stomach, in combination with the use of an active agent, that is introduced into the patient's body through a syringe, or a patch, or an inhalant or spray, or a liquid to be consumed by the patient, or a pill, or a film or a chewing gum introduced into the patient's mouth. The combination of the gastric band and the actions that introduce the active agent into the patient's body may produce a superior treatment for obesity, in comparison to a gastric band treatment alone. In one embodiment, a patient may treat obesity by performing the above-listed actions, including the use of an active agent that is introduced into the patient's body through a syringe, or a patch, or an inhalant or spray, or a liquid to be consumed by the patient, or a pill, or a film or a chewing gum introduced into the patient's mouth. The actions may be performed without the use of a gastric band.

Any of the embodiments discussed in relation to FIG. 19 may be incorporated with any other embodiment shown throughout this application. For example, a treatment involving insertion of chewing gum into the patient's mouth, may be used in combination with electric stimulation, and/or a reservoir of active agent positioned within the patient's body, and/or an ancillary device incorporated in any structure of the system. Any combination of treatments discussed throughout this application may be used as desired to enhance the treatment of obesity.

### Example of GLP-1

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing glucagon like peptide 1 (GLP-1) that is released at a rate to achieve plasma concentrations of [10-30 pMol/L]ₚ GLP-1 over a period of 3-24 months. A reservoir containing glucagon like peptide 1 (GLP-1), according to any of the embodiments of this application may also be utilized. An injection, liquid, pill, spray, inhalant, patch, or oral substance such as a gum containing glucagon like peptide (GLP-1) may also be used. The patient reports a marked suppression of appetite, and within 12 months, the patient has lost 58 pounds.

### Example of OXM

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing oxyntomodulin (OXM) that is released at a rate to achieve plasma concentrations of [105-150 pMol/L]ₚ OXM over a period of 3-24 months. A reservoir containing oxyntomodulin (OXM), according to any of the embodiments of this application may also be utilized. An injection, liquid, pill, spray, inhalant, patch, or oral substance such as a gum containing oxyntomodulin (OXM) may also be used. The patient reports a marked suppression of appetite, and within 12 months, the patient has lost 58 pounds.

### Example of PYY

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing Peptide Y-Y (PYY) that is released at a rate to achieve plasma concentrations of [10-55 pMol/L]ₚ PYY over a period of 3-24 months. A reservoir containing Peptide Y-Y (PYY), according to any of the embodiments of this application may also be utilized. An injection, liquid, pill, spray, inhalant, patch, or oral substance such as a gum containing Peptide Y-Y (PYY) may also be used. The patient reports a marked suppression of appetite, and within 12 months, the patient has lost 58 pounds.

### Example of PP

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing Pancreatic Peptide (PP) that is released at a rate to achieve plasma concentrations of [150-300 pMol/L]ₚ PP over a period of 3-24 months. A reservoir containing Pancreatic Peptide (PP), according to any of the embodiments of this application may also be utilized. An injection, liquid, pill, spray, inhalant, patch, or oral substance such as a gum containing Pancreatic Peptide (PP) may also be used. The patient reports a marked suppression of appetite, and within 12 months, the patient has lost 58 pounds.

### Example of Insulin

A 49 year old male patient, having a body weight of 322 pounds and a height of 5′11″, complains to his physician that he has tried unsuccessfully to lose weight over the past 15 years and is concerned about the effect his excess weight may have on his health. His physician suggests a procedure intended to provide the patient's body with better control over its blood sugars. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing Insulin that is released at a rate to achieve plasma concentrations of [5-30 μU/mL]ₚ Insulin over a period of 3-24 months. A reservoir containing Insulin, according to any of the embodiments of this application may also be utilized. An injection, liquid, pill, spray, inhalant, patch, or oral substance such as a gum containing Insulin may also be used. The patient reports euglycemic effects, or better control by his body of blood sugars.

### Example of Leptin

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing Leptin that is released at a rate to achieve plasma concentrations of *[3-10 ng/mL]ₚ Leptin over a period of 3-24 months. A reservoir containing Leptin, according to any of the embodiments of this application may also be utilized. An injection, liquid, pill, spray, inhalant, patch, or oral substance such as a gum containing Leptin may also be used. The patient reports a marked suppression of appetite, and within 12 months, the patient has lost 58 pounds. In the case of a female patient the goal plasma concentrations would be [10-20 ng/mL]ₚ.

### Example of Amylin

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing Amylin that is released at a rate to achieve plasma concentrations of [20-25 pMol/L]ₚ Amylin over a period of 3-24 months. A reservoir containing Amylin, according to any of the embodiments of this application may also be utilized. An injection, liquid, pill, spray, inhalant, patch, or oral substance such as a gum containing Amylin may also be used. The patient reports a marked suppression of appetite, and within 12 months, the patient has lost 58 pounds.

### Example of CCK

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing Cholecystokinin (CCK) that is released at a rate to achieve plasma concentrations of [5-10 pMol/L]ₚ CCK over a period of 3-24 months. A reservoir containing Cholecystokinin (CCK), according to any of the embodiments of this application may also be utilized. An injection, liquid, pill, spray, inhalant, patch, or oral substance such as a gum containing Cholecystokinin (CCK) may also be used. The patient reports a marked suppression of appetite, and within 12 months, the patient has lost 58 pounds.

### Example of CNTF

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing Ciliary neuro-trophic factor (CNTF) that is released at a rate to achieve plasma concentrations of [25-1300 pg/dL]ₚ CNTF over a period of 3-24 months. A reservoir containing Ciliary neuro-trophic factor (CNTF), according to any of the embodiments of this application may also be utilized. An injection, liquid, pill, spray, inhalant, patch, or oral substance such as a gum containing Ciliary neuro-trophic factor (CNTF) may also be used. The patient reports a marked suppression of appetite, and within 12 months, the patient has lost 58 pounds.

### Example of CART

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing Cocaine-Amphetamine Regulated Transcript (CART) that is released at a rate to achieve plasma concentrations of [50-250 μM]ₚ CART over a period of 3-24 months. A reservoir containing Cocaine-Amphetamine Regulated Transcript (CART), according to any of the embodiments of this application may also be utilized. An injection, liquid, pill, spray, inhalant, patch, or oral substance such as a gum containing Cocaine-Amphetamine Regulated Transcript (CART) may also be used. The patient reports a marked suppression of appetite, and within 12 months, the patient has lost 58 pounds.

### Example of Ghrelin Inhibition/Antagonism

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing a drug that is released at a rate to achieve plasma concentrations of Ghrelin at [15-30 pg/mL]ₚ over a period of 3-24 months. A reservoir containing Ghrelin blocker, according to any of the embodiments of this application may also be utilized. An injection, liquid, pill, spray, inhalant, patch, or oral substance such as a gum containing Ghrelin blocker may also be used. The patient reports a marked suppression of appetite, and within 12 months, the patient has lost 58 pounds.

### Example of NPY Inhibition/Antagonism

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing a drug that is released at a rate to achieve plasma concentrations of Neuro-peptide Y (NPY) at [65-95 pMol/L]ₚ over a period of 3-24 months. A reservoir containing Neuro-peptide Y (NPY) antagonists, according to any of the embodiments of this application may also be utilized. An injection, liquid, pill, spray, inhalant, patch, or oral substance such as a gum containing Neuro-peptide Y (NPY) antagonists may also be used. The patient reports a marked suppression of appetite, and within 12 months, the patient has lost 58 pounds.

### Example of Orexin A Inhibition/Antagonism

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing a drug that is released at a rate to achieve plasma concentrations of Orexin A at [20-50 pg/mL]ₚ over a period of 3-24 months. A reservoir containing Orexin A antagonists, according to any of the embodiments of this application may also be utilized. An injection, liquid, pill, spray, inhalant, patch, or oral substance such as a gum containing Orexin A antagonists may also be used. The patient reports a marked suppression of appetite, and within 12 months, the patient has lost 58 pounds.

### Example of AgRP Inhibition/Antagonism

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing a drug that is released at a rate to achieve plasma concentrations of AgRP at [1-16 ng/dL]ₚ over a period of 3-24 months. A reservoir containing AgRP antagonists, according to any of the embodiments of this application may also be utilized. An injection, liquid, pill, spray, inhalant, patch, or oral substance such as a gum containing AgRP antagonists may also be used. The patient reports a marked suppression of appetite, and within 12 months, the patient has lost 58 pounds.

Although the invention has been described and illustrated with a certain degree of particularity, it is to be understood that the present disclosure has been made only by way of example, and that numerous changes in the combination and arrangement of parts can be resorted to by those skilled in the art without departing from the scope of the invention, as hereinafter claimed. For example, any of the obesity treatment methods, systems, and devices discussed throughout this application may be used singularly, or in combination, as desired.

The foregoing disclosure is illustrative of the present invention and is not to be construed as limiting the invention. Although one or more embodiments of the invention have been described, persons skilled in the art will readily appreciate that numerous modifications could be made without departing from the spirit and scope of the present invention. It should be understood that all such modifications are intended to be included within the scope of the invention.

The terms “a,” “an,” “the,” and similar referents used in the context of describing the present invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the present invention otherwise claimed. No language in the specification should be construed as indicating any non-claimed element essential to the practice of the present invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, certain references have been made to patents and printed publications throughout this specification. Each of the above-cited references and printed publications are individually incorporated herein by reference in their entirety.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the present invention disclosed herein are illustrative of the principles of the present invention. Other modifications that may be employed are within the scope of the present invention. Thus, by way of example, but not of limitation, alternative configurations of the present invention may be utilized in accordance with the teachings herein. Accordingly, the present invention is not limited to that precisely as shown and described.

What is claimed is:

1. A gastric banding system configured to be laparoscopically placed around a stomach of a patient for the treatment of obesity, the gastric banding system comprising:
a gastric band configured to encircle a portion of the stomach to form a stoma;
an implantable sensor coupled to the gastric band and configured to sense a biological characteristic of the patient; and
an external control device configured to receive a telemetric signal sent in response to the biological characteristic being sensed by the implantable sensor, and to produce a notification to perform an action effective to vary the biological characteristic sensed by the implantable sensor.

2. The gastric banding system of claim 1 wherein the biological characteristic sensed by the implantable sensor is a hormone level of the patient.

3. The gastric banding system of claim 1 wherein the telemetric signal is sent telemetrically from a transmitter integrated with the implantable sensor.

4. The gastric banding system of claim 1 wherein the notification is selected from a group consisting of a visual notification, an auditory notification, a movement of the external control device, and combinations thereof.

5. The gastric banding system of claim 1 wherein the action is selected from a group consisting of injection of a hormone into the patient's body; inhalation of a hormone by the patient; drinking of a hormone by the patient; application of a patch to the patient's body being capable of distributing a hormone to the patient; spraying of a hormone into the patient's mouth; swallowing of a pill by the patient containing a hormone; insertion of a gum or film containing a hormone into the patient's mouth; and combinations thereof.

6. An implantable system for the treatment of obesity comprising:
a reservoir configured to be laparoscopically implanted into a patient's body and containing an active agent being effective, when released into the patient, to at least assist in effecting weight loss in the patient, the reservoir having an outlet configured to allow the active agent to exit the reservoir and contact a portion of the patient's body.

7. The implantable system of claim 6 wherein the reservoir is configured to be coupled to a portion of the patient's gastrointestinal tract.

8. The implantable system of claim 6 wherein the reservoir is configured to be coupled to a portion of the patient's stomach.

9. The implantable system of claim 7 wherein the reservoir is configured to encircle the portion of the patient's stomach to form a stoma.

10. The implantable system of claim 7 further comprising links configured to couple the reservoir to the portion of the patient's stomach.

11. The implantable system of claim 7 wherein the outlet includes a one-way valve.

12. The implantable system of claim 11 wherein the one-way valve is configured to allow the active agent to exit the reservoir in response to a force exerted against the reservoir by the portion of the patient's stomach.

13. The implantable system of claim 6 wherein the reservoir is coupled to a gastric band configured to encircle a portion of the patient's stomach to form a stoma.

14. The implantable system of claim 13 wherein the gastric band is a hydraulic gastric band having an internal lumen.

15. The implantable system of claim 13 wherein the gastric band is a mechanical gastric band operated by a motor.

16. The implantable system of claim 13 wherein the reservoir is positioned within the gastric band.

17. The implantable system of claim 13 wherein the reservoir is positioned exterior to and adjacent to the gastric band.

18. The implantable system of claim 13 further comprising an electrode coupled to the gastric band and configured to apply electric stimulation to a portion of the patient's body.

19. The implantable system of claim 6 further comprising a tube having a first end coupled to the outlet and a second end configured to dispense the active agent from the reservoir to the portion of the patient's body.

20. The implantable system of claim 6 wherein the outlet includes a pump.

21. The implantable system of claim 20 further comprising a sensor configured to detect a hormone level of the patient.

22. The implantable system of claim 21 wherein the sensor is configured to transmit a signal to the pump in response to a hormone level detected by the sensor.

23. The implantable system of claim 6 further comprising an active agent.

24. The implantable system of claim 23 wherein the active agent is selected from a group consisting of Glucagon-like peptide (GLP-1), Oxyntomodulin (OXM), Peptide YY (PYY), Pancreatic Polypeptide (PP), Insulin, Leptin, Gastrin, Gherlin blocker, inhibitors of DPP-IV, Amylin, Cholecystokinin (CCK), Pro-opiomelanocortin (POMC), and combinations thereof.

25. The implantable system of claim 6 wherein the reservoir has a conduit configured to allow the active agent to enter the reservoir.

26. The implantable system of claim 6 wherein the outlet of the reservoir is an outer surface of the reservoir that includes a semi-permeable membrane.

27. The implantable system of claim 26 further comprising an electrode configured to produce an electrical charge on a side of the semi-permeable membrane, to enhance or impede diffusion of the active agent through the semi-permeable membrane.

28. The implantable system of claim 26 further comprising an electrode configured to apply a voltage to the semi-permeable membrane.

29. The implantable system of claim 28 wherein the voltage causes a size of a pore of the semi-permeable membrane to vary.

30. The implantable system of claim 26 wherein the semi-permeable membrane is made from a material having a property that causes a size of a pore of the semi-permeable membrane to vary automatically in response to an environmental condition in the patient's body.

31. The implantable system of claim 26 wherein the reservoir is configured to store bacteria that produce the active agent.

32. The implantable system of claim 31 wherein the pores of the semi-permeable membrane are sized to prevent the bacteria from exiting the reservoir.

33. A method for the treatment of obesity comprising the steps of:
implanting a reservoir into a patient's body laparoscopically, the reservoir configured to contain an active agent being effective, when released into the patient, to at least assist in effecting weight loss in the patient, the reservoir having an outlet configured to allow to active agent to exit the reservoir and contact a portion of the patient's body.

34. The method of claim 33 wherein the step of implanting the reservoir into the patient's body laparoscopically includes coupling the reservoir to a portion of the patient's stomach.

35. The method of claim 34 wherein the reservoir is coupled to a gastric band configured to encircle the portion of the patient's stomach to form a stoma.

36. The method of claim 33 further comprising a step of fixing a tube extending from the reservoir to a part of the patient's body, the tube being configured to distribute the active agent from the reservoir to the portion of the patient's body.

37. The method of claim 36 wherein the part of the patient's body is a muscle wall.

38. The method of claim 33 further comprising a step of causing the active agent to exit the reservoir to contact the portion of the patient's body.

39. A method for the treatment of obesity comprising the steps of:
inserting an electrode into a patient's body laparoscopically; and
coupling the electrode to the lower third of the patient's esophagus, the electrode configured to apply electric stimulation to the lower third of the patient's esophagus,
wherein the electrode is utilized in combination with a gastric band positioned around a portion of the patient's stomach to form a stoma.

40. The method of claim 39 wherein the electric stimulation promotes a sensation of satiety for the patient.

41. The method of claim 39 wherein the electrode is coupled to the gastric band.

42. The method of claim 41 wherein the gastric band is positioned around the cardia of the patient's stomach, and the electrode extends from the gastric band to couple to the lower third of the patient's esophagus.

43. The method of claim 41 wherein the gastric band includes a control device configured to cause the electrode to apply the electric stimulation to the lower third of the patient's esophagus.

44. The method of claim 39 wherein the step of inserting the electrode into the patient's body laparoscopically further comprises inserting a plurality of electrodes into the patient's body laparoscopically, and the step of coupling the electrode to the lower third of the patient's stomach further comprises coupling the plurality of electrodes to the lower third of the patient's esophagus along the vagus nerve.
